(19) United States
(12) Patent Application Publication
(10) Pub. No.: US 2026/0201638 A1
(43) Pub. Date: Jul. 16, 2026

(54) TEXTILE DYEING

(71) Applicant: Prism Bio, Inc., Davis, CA (US)

(72) Inventors: Lauren BROERING, Woodland, CA (US); Jayarama SHETTY, Pleasanton, CA (US); Glenn NEDWIN, Davis, CA (US); Troy WILSON, Amana, IA (US)

(21) Appl. No.: 19/137,507

(22) PCT Filed: Dec. 14, 2023
(86) PCT No.: PCT/US2023/084039

§ 371 Date: Jun. 10, 2025

Related U.S. Application Data
(60) Provisional application No. 63/387,687, filed on Dec. 15, 2022.
(60) Provisional application No. 63/492,850, filed on Mar. 29, 2023.
(60) Provisional application No. 63/532,957, filed on Aug. 16, 2023.
(60) Provisional application No. 63/584,036, filed on Sep. 20, 2023.

(51) Int. Cl. D06P 5/00; A61K 8/73; A61K 8/81; A61K 8/84; A61K 8/9706; A61Q 5/10; D06P 1/44; D06P 1/46; D06P 1/48; D06P 1/52; D06P 1/54; D06P 3/04; D06P 3/52; D06P 3/60; D06P 5/04

(52) U.S. Cl. CPC D06P 5/002; A61K 8/736; A61K 8/8158; A61K 8/84; A61K 8/9706; A61Q 5/10; D06P 1/445; D06P 1/46; D06P 1/48; D06P 1/525; D06P 1/5278; D06P 1/54; D06P 3/04; D06P 3/52; D06P 3/60; D06P 5/04

(57) ABSTRACT

Disclosed herein is a textile dyeing process. The method comprises surface functionalization of a fiber using a poly-ionic polymer followed by contacting the fiber with an aqueous color solution comprising a protein bonded light harvesting chromophore. The color is then deposited and fixed on to the surface of the fiber followed by cross-linking with a bi-functional protein cross-linking agent.

### CROSS REFERENCE TO RELATED APPLICATIONS

This application claims the benefit of priority to U.S. Provisional Application No. 63/387,687 filed Dec. 15, 2022, U.S. Provisional Application No. 63/492,850 filed Mar. 29, 2023, U.S. Provisional Application No. 63/532,957 filed Aug. 16, 2023, and U.S. Provisional Application No. 63/584,036 filed Sep. 20, 2023, each of which are incorporated by reference herein in their entireties for all purposes.

### FIELD

The present disclosure provides methods and compositions to dye fiber (e.g., fabrics, hair, etc.) using environmentally sustainable methods.

### BACKGROUND

Industrial textile dyeing has several environmental and public health consequences. One of the reasons textile dyeing is environmentally unfriendly is due to achieving wash fastness, or the resistance of textiles to fading or discoloration by washing. The wash fastness of direct dye is not ideal due to the use of harsh chemicals. Dyeing procedures for vat dyes is difficult and not economical. Sulphur dye is unhygienic for the environment. Likewise, the azo dyeing procedure is complicated and time consuming. While some reactive dyes have good wash fastness properties, these dyes are only limited to cellulosic (cotton) and protein fibers (silk, wool, and jute). (R. S Chavan et. al 2011 Handbook of Textiles and Industrial dyeing).

Synthetic organic dyes come from crude oil. The dyeing process produces large Green House Gas (GHG) emission due to the high heat (>80° C.) for multiple dyeing bath steps, followed by multiple high heat washing steps. This is followed by drying, and curing at 110° C. for 2 min.

Overall, the textile dyeing industry uses some nine (9) trillion liters of water per year. Dye wastewater is one of the most harmful effluents and is carcinogenic to human and aquatic life with almost 75% of the water consumed by dye mills ends up as undrinkable waste. This is because the dyes produce harmful byproducts, due to non-specific hydrolysis of reactive dye under high alkaline conditions, resulting in a nonreactive oxi-dye form, which is lost in the dyeing process and passes into wastewater. This is responsible for the eutrophication and non-esthetic pollution from salts, alkali, heavy metals. Moreover, unfixed or hydrolyzed reactive dye must be washed off thoroughly to achieve the desired superior wet fastness of the reactive dyeing, resulting in very high levels of wastewater discharge. In addition, a significant amount (2-50%) of dyes are discharged in the water system (Mamun. M. A and Hossain M. I; 2015, 1-38; Treatment of cotton fabric with chitosan for dyeing with reactive dyes, in Textile Learner One stop solution for textile).

Natural dyes having been used since ancient times for coloring and printing fibers. Until 1896, with the discovery of oil, all textiles used throughout the world used natural dyes. Natural dyes are derived mainly from plants sources, i.e., flowers, roots, wood, leaves and few from animals/insects' sources. Additionally, blue colored pigment-proteins from blue-green microalga belonging to the light-harvesting phycobiliprotein family have been used as traditional natural food and beverage coloring ingredients as well as additives in the food, textile and cosmetic industries. Among microalgae, Spirulina maxima, Spirulina platensis and Spirulina fusiformis are the most widely cultivated species around the world and are widely used as natural pigment-protein colorants in various commercial sectors, and as supplementary additives in the food, textile, and cosmetics industries.

In addition to food, beverages, cosmetic and medical diagnostic applications of protein bonded pigments, the potential of protein bonded pigment (phycocyanin, PC) from Spirulina maxima as a source of natural dyes has been studied in recent years. Some publications and patent applications describe methods to dye textiles, such as cotton, silk, wool, jute and artificial/synthetic fabrics, etc. using phycocyanin, a natural vibrant blue color from Spirulina in a dyeing process was carried out at an ambient temperature (See Matsuo Kimura, 1993, Japanese Patent Application #07166480A; Vlakna a textile 2016, 23 (3), 56-61). However, the dyeing process described in JPH07166480 A involves soaking of the fabric in aqueous solution of phycocyanin and causing the phycocyanin to deposit and fix on to the fiber with an acid treatment. Moreover, the prior art describes chemical stabilization of the phycocyanin from Spirulina by cross-linking with formaldehyde in order to increase thermostability (See J. Biotechnol 2006, 121, 563-569). Mona, et al. (J. Algal Biomass Utln. 2019, 10, 2. 1-18) reported a method to dye the cotton fabric with selected mordants, such as alum, tannic acid, aloe vera, mango kernel powder, lemon juice. As such, while the prior art has described limited use of phycocyanin to dye fabric, all have certain shortcomings, such as the use of harsh chemicals (formaldehyde) or do not achieve adequate wash fastness.

From the many reasons discussed above, there is an unmet need to transform the textile dyeing process into an environmentally friendly process. Innovation in the textile dyeing process is needed to improve safety to the health of humans and aquatic lives by eliminating and/or reducing the need for high temperature requirements, and to reduce the discharge of wastewater containing toxic hydrolyzed byproduct from synthetic dyes.

### SUMMARY

In one aspect, provided herein is a method of dyeing a fiber, comprising: (a) contacting a fiber with a functionalizing agent comprising a poly-ionic polymer; (b) contacting the fiber with a dyeing agent comprising a protein bonded light harvesting chromophore; (c) contacting the fiber with a depositing agent; and (d) contacting the fiber with a cross-linking agent, wherein a dyed fiber is obtained. In some cases, the fiber is a natural fiber. In some cases, the fiber is a cellulosic fiber. In some cases, the fiber is a cotton, hemp, or jute fiber. In some cases, the fiber is a protein fiber. In some cases, the fiber is a silk or wool fiber. In some cases, the fiber is a synthetic fiber. In some cases, the fiber is a rayon, nylon, or polyester fiber. In some cases, the fiber is comprised within a textile. In some cases, the fiber is comprised within a fabric. In some cases, the fiber is comprised within a garment. In some cases, the fiber is hair. In some cases, the fiber is human hair. In some cases, the poly-ionic polymer comprises a poly cationic polymer. In some cases, the poly-ionic polymer comprises a poly cationic polymer comprising a positively charged functional group. In some cases, the poly-ionic polymer is selected from the group consisting of: polyethyleneimine, chitosan, derivative of polyacrylamide, carboxy methyl cellulose (CMC), and diethyl aminoethyl cellulose (DEAE-Cellulose). In some cases, the poly-ionic polymer is polyethyleneimine. In some cases, the poly-ionic polymer comprises a poly anionic polymer. In some cases, the poly-ionic polymer comprises a poly anionic polymer comprising a negatively charged functional group. In some cases, the poly-ionic polymer is selected from the group consisting of: derivative of polyacrylamide, carboxy methyl cellulose (CMC), and diethyl aminoethyl cellulose (DEAE-Cellulose). In some cases, the poly-ionic polymer is present at a concentration of about 0.01 to 20%. In some cases, the poly-ionic polymer is present at a concentration of about 0.05 to 5%. In some cases, the poly-ionic polymer is present at a concentration of about 0.2 to 0.5%. In some cases, the fiber is contacted with the functionalizing agent comprising a poly-ionic polymer for about 5 minutes or less. In some cases, the fiber is contacted with the functionalizing agent comprising a poly-ionic polymer for at least about 1 minute. In some cases, the fiber is contacted with the functionalizing agent comprising a poly-ionic polymer for at least about 10 minutes. In some cases, the fiber is contacted with the functionalizing agent comprising a poly-ionic polymer for at least about 30 minutes. In some cases, the fiber is contacted with the functionalizing agent comprising a poly-ionic polymer for at least about 60 minutes. In some cases, the fiber is contacted with the functionalizing agent comprising a poly-ionic polymer for at least about 120 minutes. In some cases, the fiber is contacted with the functionalizing agent comprising a poly-ionic polymer for at least about 24 hours. In some cases, the protein bonded light harvesting chromophore comprises a tetra pyrrole ring. In some cases, the protein bonded light harvesting chromophore comprises a modified tetra pyrrole ring. In some cases, the protein bonded light harvesting chromophore comprises a modified tetra pyrrole ring, wherein the tetra pyrrole ring has a different light harvest capacity, as compared to an unmodified tetra pyrrole ring. In some cases, the protein bonded light harvesting chromophore comprises a phycobiliprotein. In some cases, the protein bonded light harvesting chromophore is produced by a green or blue algae. In some cases, the protein bonded light harvesting chromophore is produced by a Spirulina sp. In some cases, the protein bonded light harvesting chromophore comprises a cyanobacteriochrome. In some cases, the protein bonded light harvesting chromophore is produced by a Cyanobacteria. In some cases, the depositing agent comprises an acid solution. In some cases, the depositing agent comprises an acid solution comprising tannic acid. In some cases, the depositing agent comprises an acid solution comprising citric acid. In some cases, the depositing agent comprises a mordant. In some cases, the depositing agent comprises a metal salt. In some cases, the depositing agent is present at a concentration of about 0.01 to 20%. In some cases, the depositing agent is present at a concentration of about 5% to 15%. In some cases, the cross-linking agent comprises a bi-functional protein. In some cases, the cross-linking agent comprises glutaraldehyde. In some cases, the cross-linking agent is present at a concentration of about 0.1 to 10%. In some cases, the cross-linking agent is present at a concentration of about 0.1 to 5%. In some cases, the cross-linking agent is present at a concentration of about 0.1 to 0.5%. In some cases, the method further comprises: (e) contacting the fiber with a fixing agent. In some cases, the fixing agent comprises a mordant. In some cases, the fixing agent comprises a metal salt. In some cases, the fixing agent comprises nickel sulphate. In some cases, the fixing agent comprises nickel sulphate present at a concentration of about 0.1 to 5%. In some cases, the fixing agent comprises nickel sulphate present at a concentration of about 0.1 to 1%. In some cases, the method further comprises rinsing unbound protein bonded light harvesting chromophore from the fiber. In some cases, the method further comprises repeating any of steps (a)-(e) one or more times. In some cases, steps (a)-(e) are conducted at ambient temperature. In some cases, steps (a)-(e) are conducted at a temperature of 40° C. or below. In some cases, steps (a)-(e) are conducted at a temperature of 30° C. or below. In some cases, steps (a)-(e) are conducted at a temperature of between about 15° C. to 30° C. In some cases, the dyeing agent is present at a concentration of about 0.1 to 15%. In some cases, the dyeing agent is present at a concentration of about 1 to 10%. In some cases, the dyeing agent is present at a concentration of about 5%. In some cases, the obtained dyed fiber has a stable color. In some cases, the obtained dyed fiber has a score of 62 or below on the Hunter-L scale. In some cases, the obtained dyed fiber has a stable score, as measured on the Hunter-L scale. In some cases, the obtained dyed fiber has a stable score, as measured on the Hunter-L scale, such that the dyed fiber's Hunter-L score does not fluctuate more than 10 units after washing in water. In some cases, the obtained dyed fiber has a stable score, as measured on the Hunter-L scale, such that the dyed fiber's Hunter-L score does not fluctuate more than 5 units after washing in water. In some cases, the obtained dyed fiber has a stable color, as compared to a dyed fiber that was not subject to a step (a) of contacting with a functionalizing agent comprising a poly-ionic polymer. In some cases, the method further comprises pre-wetting the fiber before step (a). In some cases, the pre-wetting comprises contacting the fiber with water.

In another aspect, provided herein is a fiber dyed by a method comprising: (a) contacting a fiber with a functionalizing agent comprising a poly-ionic polymer; (b) contacting the fiber with a dyeing agent comprising a protein bonded light harvesting chromophore; (c) contacting the fiber with a depositing agent; and (d) contacting the fiber with a cross-linking agent, wherein a dyed fiber is obtained. In some cases, the fiber is a natural fiber. In some cases, the fiber is a cellulosic fiber. In some cases, the fiber is a cotton, hemp, or jute fiber. In some cases, the fiber is a protein fiber. In some cases, the fiber is a silk or wool fiber. In some cases, the fiber is a synthetic fiber. In some cases, the fiber is a rayon, nylon, or polyester fiber. In some cases, the fiber is hair. In some cases, the fiber is human hair. In some cases, the fiber is comprised within a textile. In some cases, the fiber is comprised within a fabric. In some cases, the fiber is comprised within a garment. In some cases, the poly-ionic polymer comprises a poly cationic polymer. In some cases, the poly-ionic polymer comprises a poly cationic polymer comprising a positively charged functional group. In some cases, the poly-ionic polymer is selected from the group consisting of: polyethyleneimine, chitosan, derivative of polyacrylamide, carboxy methyl cellulose (CMC), and diethyl aminoethyl cellulose (DEAE-Cellulose). In some cases, the poly-ionic polymer is polyethyleneimine. In some cases, the poly-ionic polymer comprises a poly anionic polymer. In some cases, the poly-ionic polymer comprises a poly anionic polymer comprising a negatively charged functional group. In some cases, the poly-ionic polymer is selected from the group consisting of: derivative of polyacrylamide, carboxy methyl cellulose (CMC), and diethyl aminoethyl cellulose (DEAE-Cellulose). In some cases, the poly-ionic polymer is present at a concentration of about 0.01 to 20%. In some cases, the poly-ionic polymer is present at a concentration of about 0.05 to 5%. In some cases, the poly-ionic polymer is present at a concentration of about 0.2 to 0.5%. In some cases, the fiber is contacted with the functionalizing agent comprising a poly-ionic polymer for about 5 minutes or less. In some cases, the fiber is contacted with the functionalizing agent comprising a poly-ionic polymer for at least about 1 minute. In some cases, the fiber is contacted with the functionalizing agent comprising a poly-ionic polymer for at least about 10 minutes. In some cases, the fiber is contacted with the functionalizing agent comprising a poly-ionic polymer for at least about 30 minutes. In some cases, the fiber is contacted with the functionalizing agent comprising a poly-ionic polymer for at least about 60 minutes. In some cases, the fiber is contacted with the functionalizing agent comprising a poly-ionic polymer for at least about 120 minutes. In some cases, the fiber is contacted with the functionalizing agent comprising a poly-ionic polymer for at least about 24 hours. In some cases, the protein bonded light harvesting chromophore comprises a tetra pyrrole ring. In some cases, the protein bonded light harvesting chromophore comprises a modified tetra pyrrole ring. In some cases, the protein bonded light harvesting chromophore comprises a modified tetra pyrrole ring, wherein the tetra pyrrole ring has a different light harvest capacity, as compared to an unmodified tetra pyrrole ring. In some cases, the protein bonded light harvesting chromophore comprises a phycobiliprotein. In some cases, the protein bonded light harvesting chromophore is produced by a green or blue algae. In some cases, the protein bonded light harvesting chromophore is produced by a Spirulina sp. In some cases, the protein bonded light harvesting chromophore comprises a cyanobacteriochrome. In some cases, the protein bonded light harvesting chromophore is produced by a Cyanobacteria. In some cases, the depositing agent comprises an acid solution. In some cases, the depositing agent comprises an acid solution comprising tannic acid. In some cases, the depositing agent comprises an acid solution comprising citric acid. In some cases, the depositing agent comprises a mordant. In some cases, the depositing agent comprises a metal salt. In some cases, the depositing agent is present at a concentration of about 0.01 to 20%. In some cases, the depositing agent is present at a concentration of about 5% to 15%. In some cases, the cross-linking agent comprises a bi-functional protein. In some cases, the cross-linking agent comprises glutaraldehyde. In some cases, the cross-linking agent is present at a concentration of about 0.1 to 10%. In some cases, the cross-linking agent is present at a concentration of about 0.1 to 5%. In some cases, the cross-linking agent is present at a concentration of about 0.1 to 0.5%. In some cases, the method further comprises: (e) contacting the fiber with a fixing agent. In some cases, the fixing agent comprises a mordant. In some cases, the fixing agent comprises a metal salt. In some cases, the fixing agent comprises nickel sulphate. In some cases, the fixing agent comprises nickel sulphate present at a concentration of about 0.1 to 5%. In some cases, the fixing agent comprises nickel sulphate present at a concentration of about 0.1 to 1%. In some cases, the method further comprises rinsing unbound protein bonded light harvesting chromophore from the fiber. In some cases, the method further comprises repeating any of steps (a)-(e) one or more times. In some cases, steps (a)-(e) are conducted at ambient temperature. In some cases, steps (a)-(e) are conducted at a temperature of 40° C. or below. In some cases, steps (a)-(e) are conducted at a temperature of 30° C. or below. In some cases, steps (a)-(e) are conducted at a temperature of between about 15° C. to 30° C. In some cases, the dyeing agent is present at a concentration of about 0.1 to 15%. In some cases, the dyeing agent is present at a concentration of about 1 to 10%. In some cases, the dyeing agent is present at a concentration of about 5%. In some cases, the obtained dyed fiber has a stable color. In some cases, the obtained dyed fiber has a score of 62 or below on the Hunter-L scale. In some cases, the obtained dyed fiber has a stable score, as measured on the Hunter-L scale. In some cases, the obtained dyed fiber has a stable score, as measured on the Hunter-L scale, such that the dyed fiber's Hunter-L score does not fluctuate more than 10 units after washing in water. In some cases, the obtained dyed fiber has a stable score, as measured on the Hunter-L scale, such that the dyed fiber's Hunter-L score does not fluctuate more than 5 units after washing in water. In some cases, the obtained dyed fiber has a stable color, as compared to a dyed fiber that was not subject to a step (a) of contacting with a functionalizing agent comprising a poly-ionic polymer. In some cases, the method further comprises pre-wetting the fiber before step (a). In some cases, the pre-wetting comprises contacting the fiber with water.

In still another aspect, provided herein is a thread, woven thread, non-woven thread, yarn, textile, fabric or garment comprising a dyed fiber produced by a method provided herein. In some cases, the method comprises: (a) contacting a fiber with a functionalizing agent comprising a poly-ionic polymer; (b) contacting the fiber with a dyeing agent comprising a protein bonded light harvesting chromophore; (c) contacting the fiber with a depositing agent; and (d) contacting the fiber with a cross-linking agent, wherein a dyed fiber is obtained. In some cases, the fiber is a natural fiber. In some cases, the fiber is a cellulosic fiber. In some cases, the fiber is a cotton, hemp, or jute fiber. In some cases, the fiber is a protein fiber. In some cases, the fiber is a silk or wool fiber. In some cases, the fiber is a synthetic fiber. In some cases, the fiber is a rayon, nylon, or polyester fiber. In some cases, the poly-ionic polymer comprises a poly cationic polymer. In some cases, the poly-ionic polymer comprises a poly cationic polymer comprising a positively charged functional group. In some cases, the poly-ionic polymer is selected from the group consisting of: polyethyleneimine, chitosan, derivative of polyacrylamide, carboxy methyl cellulose (CMC), and diethyl aminoethyl cellulose (DEAE-Cellulose). In some cases, the poly-ionic polymer is polyethyleneimine. In some cases, the poly-ionic polymer comprises a poly anionic polymer. In some cases, the poly-ionic polymer comprises a poly anionic polymer comprising a negatively charged functional group. In some cases, the poly-ionic polymer is selected from the group consisting of: derivative of polyacrylamide, carboxy methyl cellulose (CMC), and diethyl aminoethyl cellulose (DEAE-Cellulose). In some cases, the poly-ionic polymer is present at a concentration of about 0.01 to 20%. In some cases, the poly-ionic polymer is present at a concentration of about 0.05 to 5%. In some cases, the poly-ionic polymer is present at a concentration of about 0.2 to 0.5%. In some cases, the fiber is contacted with the functionalizing agent comprising a poly-ionic polymer for about 5 minutes or less. In some cases, the fiber is contacted with the functionalizing agent comprising a poly-ionic polymer for at least about 1 minute. In some cases, the fiber is contacted with the functionalizing agent comprising a poly-ionic polymer for at least about 10 minutes. In some cases, the fiber is contacted with the functionalizing agent comprising a poly-ionic polymer for at least about 30 minutes. In some cases, the fiber is contacted with the functionalizing agent comprising a poly-ionic polymer for at least about 60 minutes. In some cases, the fiber is contacted with the functionalizing agent comprising a poly-ionic polymer for at least about 120 minutes. In some cases, the fiber is contacted with the functionalizing agent comprising a poly-ionic polymer for at least about 24 hours. In some cases, the protein bonded light harvesting chromophore comprises a tetra pyrrole ring. In some cases, the protein bonded light harvesting chromophore comprises a modified tetra pyrrole ring. In some cases, the protein bonded light harvesting chromophore comprises a modified tetra pyrrole ring, wherein the tetra pyrrole ring has a different light harvest capacity, as compared to an unmodified tetra pyrrole ring. In some cases, the protein bonded light harvesting chromophore comprises a phycobiliprotein. In some cases, the protein bonded light harvesting chromophore is produced by a green or blue algae. In some cases, the protein bonded light harvesting chromophore is produced by a Spirulina sp. In some cases, the protein bonded light harvesting chromophore comprises a cyanobacteriochrome. In some cases, the protein bonded light harvesting chromophore is produced by a Cyanobacteria. In some cases, the depositing agent comprises an acid solution. In some cases, the depositing agent comprises an acid solution comprising tannic acid. In some cases, the depositing agent comprises an acid solution comprising citric acid. In some cases, the depositing agent comprises a mordant. In some cases, the depositing agent comprises a metal salt. In some cases, the depositing agent is present at a concentration of about 0.01 to 20%. In some cases, the depositing agent is present at a concentration of about 5% to 15%. In some cases, the cross-linking agent comprises a bi-functional protein. In some cases, the cross-linking agent comprises glutaraldehyde. In some cases, the cross-linking agent is present at a concentration of about 0.1 to 10%. In some cases, the cross-linking agent is present at a concentration of about 0.1 to 5%. In some cases, the cross-linking agent is present at a concentration of about 0.1 to 0.5%. In some cases, the method further comprises: (e) contacting the fiber with a fixing agent. In some cases, the fixing agent comprises a mordant. In some cases, the fixing agent comprises a metal salt. In some cases, the fixing agent comprises nickel sulphate. In some cases, the fixing agent comprises nickel sulphate present at a concentration of about 0.1 to 5%. In some cases, the fixing agent comprises nickel sulphate present at a concentration of about 0.1 to 1%. In some cases, the method further comprises rinsing unbound protein bonded light harvesting chromophore from the fiber. In some cases, the method further comprises repeating any of steps (a)-(e) one or more times. In some cases, steps (a)-(e) are conducted at ambient temperature. In some cases, steps (a)-(e) are conducted at a temperature of 40° C. or below. In some cases, steps (a)-(e) are conducted at a temperature of 30° C. or below. In some cases, steps (a)-(e) are conducted at a temperature of between about 15° C. to 30° C. In some cases, the dyeing agent is present at a concentration of about 0.1 to 15%. In some cases, the dyeing agent is present at a concentration of about 1 to 10%. In some cases, the dyeing agent is present at a concentration of about 5%. In some cases, the obtained dyed fiber has a stable color. In some cases, the obtained dyed fiber has a score of 62 or below on the Hunter-L scale. In some cases, the obtained dyed fiber has a stable score, as measured on the Hunter-L scale. In some cases, the obtained dyed fiber has a stable score, as measured on the Hunter-L scale, such that the dyed fiber's Hunter-L score does not fluctuate more than 10 units after washing in water. In some cases, the obtained dyed fiber has a stable score, as measured on the Hunter-L scale, such that the dyed fiber's Hunter-L score does not fluctuate more than 5 units after washing in water. In some cases, the obtained dyed fiber has a stable color, as compared to a dyed fiber that was not subject to a step (a) of contacting with a functionalizing agent comprising a poly-ionic polymer. In some cases, the method further comprises pre-wetting the fiber before step (a). In some cases, the pre-wetting comprises contacting the fiber with water.

In another aspect, provided herein is a composition comprising a fiber, wherein the fiber has a protein bonded light harvesting chromophore and a cross-linking agent attached thereto. In some cases, the protein bonded light harvesting chromophore comprises a tetra pyrrole ring. In some cases, the protein bonded light harvesting chromophore comprises a modified tetra pyrrole ring. In some cases, the protein bonded light harvesting chromophore comprises a modified tetra pyrrole ring, wherein the tetra pyrrole ring has a different light harvest capacity, as compared to an unmodified tetra pyrrole ring. In some cases, the protein bonded light harvesting chromophore comprises a phycobiliprotein. In some cases, the protein bonded light harvesting chromophore is produced by a green or blue algae. In some cases, the protein bonded light harvesting chromophore is produced by a Spirulina sp. In some cases, the protein bonded light harvesting chromophore comprises a cyanobacteriochrome. In some cases, the protein bonded light harvesting chromophore is produced by a Cyanobacteria. In some cases, the cross-linking agent comprises a bi-functional protein. In some cases, the cross-linking agent comprises glutaraldehyde. In some cases, the cross-linking agent is bonded with primary, secondary, or tertiary amino groups.

In another aspect, provided herein is a method for dyeing hair as taught herein. In some cases, the hair is human hair.

### BRIEF DESCRIPTION OF THE FIGURES

FIG. 1A shows a general schematic flow diagram of an ambient temperature textile dyeing process using spirulina.

FIG. 1B shows an example of a flow diagram with incubation times, pH and reagent concentrations for an ambient temperature dyeing of cotton fabric using Spirulina by the process of the present disclosure.

FIG. 2A shows images of the effect of soaking time of cotton fabric in 0.2% polyethyleneimine and water on the darkness of spirulina (5%) dyed cotton fabric.

FIG. 2B shows images of the effect of cold and hot washes with commercial detergent of spirulina dyed cotton fabric soaked in water without added PEI.

FIG. 3A shows graphically, the effect of PEI concentration during soaking of cotton fabric on the darkness of spirulina dyed fabric before and after cold and hot washes.

FIG. 3B shows images of the effect of PEI concentration during soaking of cotton fabric on the darkness of spirulina dyed cotton fabric before and after cold and hot washes in comparison to unwashed, dyed controls.

FIG. 4A show images of the effect on final darkness of dyed fabric following repeated dyeing of 0.2% PEI-soaked cotton fabric with Spirulina using ambient temperature dyeing process of the present disclosure.

FIG. 4B shows graphically, the effect on final darkness of dyed fabric following repeated dyeing of 0.2% PEI-soaked cotton with spirulina on the darkness of dyed cotton fabric using ambient temperature dyeing process of the present disclosure.

FIG. 5 shows images of the effect of repeated cold and hot wash using commercial detergent product of spirulina dyed cotton fabric (5% PEI soaked) on the darkness of dried cotton fabric.

FIG. 6 shows images of the effect of glutaraldehyde (GA) concentration during ambient temperature spirulina dyeing process on the darkness of 5% PEI-soaked cotton fabric before and after cold and hot washes.

FIGS. 7A and 7B show images of the effect of ambient temperature textile dyeing process using protein fabric; i.e., Silk.

FIG. 8 shows images of the effect of Chitosan soaking of cotton fabric on the darkness of spirulina dyed cotton fabric, before and after cold and hot wash.

FIG. 9 shows images of the effect of a poly anionic polymer, Tramfloc® 146, soaking of cotton fabric on the darkness of spirulina dyed cotton fabric before and after cold and hot washes.

FIG. 10A shows an image of the effect of ambient temperature textile dyeing process using synthetic fabric; i.e., polyester.

FIGS. 10B and 10C show images of the effect of ambient temperature textile dyeing process using synthetic fabric; i.e., polyester, after a first hot water wash and a second hot water wash, respectively.

FIG. 11 shows a general schematic flow diagram of an ambient temperature textile dyeing process using spirulina.

FIG. 12 shows images of the effect of the scaled-up ambient temperature textile dyeing process using cotton fabric.

FIG. 13 shows images of the effect of the scaled-up ambient temperature textile dyeing process using cotton fabric after one and two hot washes.

FIG. 14 shows the image of a PEI-Spirulina dyed hair swatches either undyed or dyed with either (1) PEI-Spirulina, (2) PEI-Spirulina+tannic acid, or (3) PEI-Spirulina+tannic acid+glutaraldehyde.

### DETAILED DESCRIPTION

### Definitions

As used herein, the singular forms “a”, “an” and “the” include plural referents unless the context clearly dictates otherwise.

All embodiments of any aspect of the disclosure can be used in combination unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Additionally, the words “herein,” “above,” “below,” and words of similar import, when used in this application, shall refer to this application as a whole and not to any particular portions of the application.

As used herein, the term “natural pigments” can refer to plant extract from leaf, roots, and fruits belonging to carotenoids, anthocyanin, beta carotene, Zeaxanthin, and the color pigments produced by microorganisms.

The term “pigment-protein” can refer to proteins bound to a light harvesting chromophore, i.e., bilin, which results in the emission of various UV-Visible colors.

The term “Cyanobacteriochrome” or “CBCR” can refer to linear tetrapyrrole containing proteins or biliproteins.

The term “bilins” can refer to light-absorbing prosthetic groups that are found bound to proteins.

The term “phycobiliproteins” can refer to a class of proteins with shared sequence similarities conserved bilin-binding residues.

The term “phycocyanin” can refer to a pigment protein-complex from light harvesting phycobiliproteins family.

The term “mordant” can refer to a substance used to fix a dye to fabric.

The term “Purity Index (PI) Ratio” can refer to the ratio of Optical density at maximum absorption in the visible region to the optical density at 280 nm of a protein bonded light harvesting chromophore.

Hunter L, a, b can refer to both color scale measurements based on the opponent-color theory. This theory assumes that the receptors in the human eye perceive color the following pairs of opposite (Hunter Lab: Application Note #AN 1005.00).

“L” scale can refer to Light vs Dark where a low number (0-50) indicates dark, and a high number (50-100) indicates light.

“a” scale can refer to red vs green where a positive number indicates red, and a negative number indicates green.

“b” scale can refer to yellow vs blue where a positive number indicates yellow, and a negative number indicates blue.

The term “contacting” can refer to the placing the protein bonded pigment to enable to interact with surface functionalized cotton/protein fiber.

The term “OD” can refer to optical density measured at a specified wavelength of a clear solution.

The term “surface functionalization” can refer to the act of modifying a surface to give it physical, chemical, or biological characteristics different from the original.

The term “fiber” can refer to, but is not limited to, a natural fiber, a cellulosic fiber, cotton fiber, hemp fiber, jute fiber, protein fiber, silk fiber, wool fiber, synthetic fiber, rayon fiber, nylon fiber, and/or polyester fiber. In some embodiments, a fiber is comprised within a textile. In some embodiments, a fiber is comprised within a fabric. In some embodiments, a fiber is comprised within a garment.

The terms “scaling up” or “scaled-up” can refer to, but may not be limited to, increasing the production capacity of the methods and compositions of the disclosure.

### Overview

The current disclosure solves the aforementioned problems through the creation and utilization of methods to dye fabric using environmentally sustainable methods. The methods of the present disclosure do not use high temperatures and do not produce toxic byproducts.

Thus, in some embodiments, the present disclosure teaches a method of dyeing a fiber, comprising contacting a fiber with a functionalizing agent, which can comprise a poly-ionic polymer; contacting the fiber with a dyeing agent, which can comprise a protein bonded light harvesting chromophore; contacting the fiber with a depositing agent; and contacting the fiber with a cross-linking agent. The fiber can be a natural fiber, a synthetic fiber, a protein fiber or a cellulosic fiber. In some cases, the fiber is a cotton, hemp, or jute fiber. In some cases, the fiber is a silk or wool fiber. In some cases, the fiber is a rayon, nylon, or polyester fiber. In some cases, the fiber is comprised within a textile. In some cases, the fiber is comprised within a fabric. In some cases, the fiber is comprised within a garment. The functionalizing agent can be any such agent known in the art and/or provided herein. The dyeing agent can be any such agent known in the art and/or provided herein. The cross-linking agent can be any such agent known in the art and/or provided herein. The depositing agent can be any such agent known in the art and/or provided herein. In some cases, the method can further comprise contacting the fiber with a fixing agent. The fixing agent can be a mordant. The mordant can be any mordant known in the art. The fixing agent can be a metal salt. In some cases, the fixing agent can be nickel sulphate. In some cases, the method can further comprise rinsing any unbound dyeing agent (e.g., a protein bonded light harvesting chromophore). In some cases, the method can further comprise repeating any of aforementioned steps one or more times. In some cases, one or more of the aforementioned steps can be conducted at ambient temperature. In some cases, one or more of the aforementioned steps can be conducted at a temperature of 40° C. or below. In some cases, one or more of the aforementioned steps can be conducted at a temperature of 30° C. or below. In some cases, one or more of the aforementioned steps can be conducted at a temperature of between about 15° C. to 30° C. In some embodiments, a dyed fiber is obtained.

In some embodiments, the present disclosure also teaches a composition for use in dyeing a fiber according to the methods provided herein. The composition can comprise a fiber, wherein the fiber has a protein bonded light harvesting chromophore and a cross-linking agent attached thereto. The protein bonded light harvesting chromophore can be any such chromophore known in the art and/or provided herein. In some cases, the protein bonded light harvesting chromophore comprises a tetra pyrrole ring. In some cases, the protein bonded light harvesting chromophore comprises a modified tetra pyrrole ring. In some cases, the protein bonded light harvesting chromophore comprises a modified tetra pyrrole ring, wherein the tetra pyrrole ring has a different light harvest capacity, as compared to an unmodified tetra pyrrole ring. In some cases, the protein bonded light harvesting chromophore comprises a phycobiliprotein. In some cases, the protein bonded light harvesting chromophore is produced by a green or blue algae. In some cases, the protein bonded light harvesting chromophore is produced by a Spirulina sp. In some cases, the protein bonded light harvesting chromophore comprises a cyanobacteriochrome. In some cases, the protein bonded light harvesting chromophore is produced by a Cyanobacteria. The protein cross-linking agent can be any such cross-linking agent known in the art and/or provided herein. In some cases, the cross-linking agent comprises a bi-functional protein. In some cases, the cross-linking agent comprises glutaraldehyde.

### Poly-Ionic Polymer

In some embodiments, the fiber is contacted with a functionalizing agent. In some embodiments, the functionalizing agent comprises a poly-ionic polymer. In some embodiments, the poly-ionic polymer comprises a poly cationic polymer. In some embodiments, the poly-ionic polymer comprises a poly cationic polymer comprising a positively charged functional group. In some embodiments, the poly-ionic polymer is selected from the group consisting of: polyethyleneimine, chitosan, and/or a derivative of polyacrylamide, the poly-ionic polymer is polyethyleneimine. In some embodiments, the poly-ionic polymer comprises a poly anionic polymer. In some embodiments, the poly-ionic polymer comprises a poly anionic polymer comprising a negatively charged functional group In some embodiments, the poly anionic polymer is a derivative of polyacrylamide.

In some embodiments, the poly-ionic polymer is present at a concentration of about 0.0001%, about 0.001%, about 0.01%, about 0.05%, about 0.1%, about 0.15%, about 0.2%, about 0.25% about 0.3%, about 0.35%, about 0.4%, about 0.45%, about 0.5%, about 0.6%, about 0.7%, about 0.8%, about 0.9%, about 1%, about 2%, about 3%, about 4%, about 5%, about 6%, about 7%, about 8%, about 9%, about 10%, 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%.

In some embodiments, the poly-ionic polymer is present at a concentration of at least, at most or exactly 0.0001%, 0.001%, 0.01%, 0.05%, 0.1%, 0.15%, 0.2%, 0.25%, 0.3%, 0.35%, 0.4%, 0.45%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30%.

In some embodiments, the poly-ionic polymer is present at a concentration of from 0.0001% to about 0.001%, about 0.001% to about 0.01%, about 0.01% to about 0.1%, about 0.1% to about 0.1%, about 0.1% to about 0.2%, about 0.2% to about 0.3%, about 0.3% to about 0.4%, about 0.4% to about 0.5%, about 0.5% to about 0.6%, about 0.6% to about 0.7%, about 0.7% to about 0.8%, about 0.8% to about 0.9%, about 0.9% to about 1%, about 1% to about 2%, about 2% to about 3%, about 3% to about 4%, about 4% to about 5%, about 5% to about 6%, about 6% to about 7%, about 7% to about 8%, about 8% to about 9%, about 9% to about 10%, about 10% to about 11%, about 11% to about 12%, about 12% to about 13%, about 13% to about 14%, about 14% to about 15%, about 15% to about 16%, about 16% to about 17%, about 17% to about 18%, about 18% to about 19%, about 19% to about 20%, about 20% to about 21%, about 21% to about 22%, about 22% to about 23%, about 23% to about 24%, about 24% to about 25%, about 25% to about 26%, about 26% to about 27%, about 27% to about 28%, about 28% to about 29%, or about 29% to about 30%.

In some embodiments, the poly-ionic polymer is present at a concentration of between 0.0001%-0.001%, between 0.001%-0.01%, between 0.01%-0.1%, between 0.1%-0.1%, between 0.1%-0.2%, between 0.2%-0.3%, between 0.3%-0.4%, between 0.4%-0.5%, between 0.5%-0.6%, between 0.6%-0.7%, between 0.7%-0.8%, between 0.8%-0.9%, between 0.9%-1%, between 1%-2%, between 2%-3%, between 3%-4%, between 4%-5%, between 5%-6%, between 6%-7%, between 7%-8%, between 8%-9%, between 9%-10%, between 10%-11%, between 11%-12%, between 12%-13%, between 13%-14%, between 14%-15%, between 15%-16%, between 16%-17%, between 17%-18%, between 18%-19%, between 19%-20%, between 20%-21%, between 21%-22%, between 22%-23%, between 23%-24%, between 24%-25%, between 25%-26%, between 26%-27%, between 27%-28%, between 28%-29%, or between 29%-30%.

In some embodiments, the fiber is contacted with the functionalizing agent comprising a poly-ionic polymer. In some embodiments, the fiber is contacted with the functionalizing agent comprising a poly-ionic polymer for 1 second, 10 seconds, 30 seconds, 1 minute, 10 minutes, 20 minutes, 30 minutes, 40 minutes, 50 minutes, 1 hour, 2 hours, 3 hours, 4 hours, 8 hours, 12 hours, 24 hours, 48 hours, or 72 hours.

In some embodiments, the fiber is contacted with the functionalizing agent comprising a poly-ionic polymer. In some embodiments, the fiber is contacted with the functionalizing agent comprising a poly-ionic polymer for about 1 second, about 10 seconds, about 30 seconds, about 1 minute, about 10 minutes, about 20 minutes, about 30 minutes, about 40 minutes, about 50 minutes, about 1 hour, about 2 hours, about 3 hours, about 4 hours, about 8 hours, about 12 hours, about 24 hours, about 48 hours, or about 72 hours.

In some embodiments, the fiber is contacted with the functionalizing agent comprising a poly-ionic polymer for from about 1 second to from about 10 seconds, from about 10 seconds to from about 30 seconds, from about 30 seconds to from about 1 minute, from about 1 minute to from about 10 minute, from about 10 minutes to from about 20 minutes, from about 20 minutes to from about 30 minutes, from about 30 minutes to from about 40 minutes, from about 40 minutes to from about 50 minutes, from about 50 minutes to from about 1 hour, from about 1 hour to from about 2 hours, from about 2 hours to from about 3 hours, from about 3 hours to from about 4 hours, from about 4 hours to from about 8 hours, from about 8 hours to from about 12 hours, from about 12 hours to from about 24 hours, from about 24 hours to from about 48 hours, or from about 48 hours to about 72 hours.

In some embodiments, the fiber is contacted with the functionalizing agent comprising a poly-ionic polymer for between 1 second to 10 seconds, between 10 seconds to 30 seconds, between 30 seconds to 1 minute, between 1 minute to 10 minutes, between 10 minutes to 20 minutes, between 20 minutes to 30 minutes, between 30 minutes to 40 minutes, between 40 minutes to 50 minutes, between 50 minutes to 1 hour, between 1 hour to 2 hours, between 2 hours to 3 hours, between 3 hours to 4 hours, between 4 hours to 8 hours, between 8 hours to 12 hours, between 12 hours to 24 hours, between 24 hours to 48 hours, or between 48 hours to 72 hours.

In some embodiments, the functionalizing agent comprises a pH of about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, about 12.0, about 12.1, about 12.2, about 12.3, about 12.4, about 12.5, about 12.6, about 12.7, about 12.8, about 12.9, or about 13.0.

In some embodiments, the functionalizing agent comprises a pH of at least, at most or exactly 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, or 13.0.

### Depositing Agent

In some embodiments, the fiber is contacted with a depositing agent. In some embodiments, the depositing agent comprises an acid solution. In some embodiments, the depositing agent comprises an acid solution comprising tannic acid. In some embodiments, the depositing agent comprises an acid solution comprising citric acid. In some embodiments, the depositing agent comprises a mordant. In some embodiments, the depositing agent comprises a metal salt.

In some embodiments, the depositing agent is present at a concentration of about 0.0001%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%.

In some embodiments, the depositing agent is present at a concentration of at least, at most or exactly 0.0001%, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30%.

In some embodiments, the depositing agent is present at a concentration of from 0.0001% to about 0.001%, about 0.001% to about 0.01%, about 0.01% to about 0.1%, about 0.1% to about 0.1%, about 0.1% to about 0.2%, about 0.2% to about 0.3%, about 0.3% to about 0.4%, about 0.4% to about 0.5%, about 0.5% to about 0.6%, about 0.6% to about 0.7%, about 0.7% to about 0.8%, about 0.8% to about 0.9%, about 0.9% to about 1%, about 1% to about 2%, about 2% to about 3%, about 3% to about 4%, about 4% to about 5%, about 5% to about 6%, about 6% to about 7%, about 7% to about 8%, about 8% to about 9%, about 9% to about 10%, about 10% to about 11%, 11% to about 12%, about 12% to about 13%, about 13% to about 14%, about 14% to about 15%, about 15% to about 16%, about 16% to about 17%, about 17% to about 18%, about 18% to about 19%, about 19% to about 20%, about 20% to about 21%, about 21% to about 22%, about 22% to about 23%, about 23% to about 24%, about 24% to about 25%, about 25% to about 26%, about 26% to about 27%, about 27% to about 28%, about 28% to about 29%, or about 29% to about 30%.

In some embodiments, the depositing agent is present at a concentration of between 0.0001%-0.001%, between 0.001%-0.01%, between 0.01%-0.1%, between 0.1%-0.1%, between 0.1%-0.2%, between 0.2%-0.3%, between 0.3%-0.4%, between 0.4%-0.5%, between 0.5%-0.6%, between 0.6%-0.7%, between 0.7%-0.8%, between 0.8%-0.9%, between 0.9%-1%, between 1%-2%, between 2%-3%, between 3%-4%, between 4%-5%, between 5%-6%, between 6%-7%, between 7%-8%, between 8%-9%, between 9%-10%, between 10%-11%, between 11%-12%, between 12%-13%, between 13%-14%, between 14%-15%, between 15%-16%, between 16%-17%, between 17%-18%, between 18%-19%, between 19%-20%, between 20%-21%, between 21%-22%, between 22%-23%, between 23%-24%, between 24%-25%, between 25%-26%, between 26%-27%, between 27%-28%, between 28%-29%, or between 29%-30%.

In some embodiments, the fiber is contacted with the depositing agent. In some embodiments, the fiber is contacted with the depositing agent for about 1 second, about 10 seconds, about 30 seconds, about 1 minute, about 10 minutes, about 20 minutes, about 30 minutes, about 40 minutes, about 50 minutes, about 1 hour, about 2 hours, about 3 hours, about 4 hours, about 8 hours, about 12 hours, about 24 hours, about 48 hours, or about 72 hours.

In some embodiments, the fiber is contacted with the depositing agent for 1 second, 10 seconds, 30 seconds, 1 minute, 10 minutes, 20 minutes, 30 minutes, 40 minutes, 50 minutes, 1 hour, 2 hours, 3 hours, 4 hours, 8 hours, 12 hours, 24 hours, 48 hours, or 72 hours.

In some embodiments, the fiber is contacted with the depositing agent for from about 1 second to from about 10 seconds, from about 10 seconds to from about 30 seconds, from about 30 seconds to from about 1 minute, from about 1 minute to from about 10 minute, from about 10 minutes to from about 20 minutes, from about 20 minutes to from about 30 minutes, from about 30 minutes to from about 40 minutes, from about 40 minutes to from about 50 minutes, from about 50 minutes to from about 1 hour, from about 1 hour to from about 2 hours, from about 2 hours to from about 3 hours, from about 3 hours to from about 4 hours, from about 4 hours to from about 8 hours, from about 8 hours to from about 12 hours, from about 12 hours to from about 24 hours, from about 24 hours to from about 48 hours, or from about 48 hours to about 72 hours.

In some embodiments, the fiber is contacted with the depositing agent for between 1 second to 10 seconds, between 10 seconds to 30 seconds, between 30 seconds to 1 minute, between 1 minute to 10 minutes, between 10 minutes to 20 minutes, between 20 minutes to 30 minutes, between 30 minutes to 40 minutes, between 40 minutes to 50 minutes, between 50 minutes to 1 hour, between 1 hour to 2 hours, between 2 hours to 3 hours, between 3 hours to 4 hours, between 4 hours to 8 hours, between 8 hours to 12 hours, between 12 hours to 24 hours, between 24 hours to 48 hours, or between 48 hours to 72 hours.

In some embodiments, the depositing agent comprises a pH of about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, about 12.0, about 12.1, about 12.2, about 12.3, about 12.4, about 12.5, about 12.6, about 12.7, about 12.8, about 12.9, or about 13.0.

In some embodiments, the depositing agent comprises a pH of at least, at most or exactly 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, or 13.0.

### Cross-Linking Agent

In some embodiments, the fiber is contacted with a cross-linking agent. In some embodiments, the cross-linking agent is bi-functional. In some embodiments, the cross-linking agent comprises glutaraldehyde (GA).

In some embodiments, the cross-linking agent is present at a concentration of about 0.0001%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%.

In some embodiments, the cross-linking agent is present at a concentration of at least, at most or exactly 0.0001%, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30%.

In some embodiments, the cross-linking agent is present at a concentration of from 0.0001% to about 0.001%, about 0.001% to about 0.01%, about 0.01% to about 0.1%, about 0.1% to about 0.1%, about 0.1% to about 0.2%, about 0.2% to about 0.3%, about 0.3% to about 0.4%, about 0.4% to about 0.5%, about 0.5% to about 0.6%, about 0.6% to about 0.7%, about 0.7% to about 0.8%, about 0.8% to about 0.9%, about 0.9% to about 1%, about 1% to about 2%, about 2% to about 3%, about 3% to about 4%, about 4% to about 5%, about 5% to about 6%, about 6% to about 7%, about 7% to about 8%, about 8% to about 9%, about 9% to about 10%, about 10% to about 11%, 11% to about 12%, about 12% to about 13%, about 13% to about 14%, about 14% to about 15%, about 15% to about 16%, about 16% to about 17%, about 17% to about 18%, about 18% to about 19%, about 19% to about 20%, about 20% to about 21%, about 21% to about 22%, about 22% to about 23%, about 23% to about 24%, about 24% to about 25%, about 25% to about 26%, about 26% to about 27%, about 27% to about 28%, about 28% to about 29%, or about 29% to about 30%.

In some embodiments, the cross-linking agent is present at a concentration of between 0.0001%-0.001%, between 0.001%-0.01%, between 0.01%-0.1%, between 0.1%-0.1%, between 0.1%-0.2%, between 0.2%-0.3%, between 0.3%-0.4%, between 0.4%-0.5%, between 0.5%-0.6%, between 0.6%-0.7%, between 0.7%-0.8%, between 0.8%-0.9%, between 0.9%-1%, between 1%-2%, between 2%-3%, between 3%-4%, between 4%-5%, between 5%-6%, between 6%-7%, between 7%-8%, between 8%-9%, between 9%-10%, between 10%-11%, between 11%-12%, between 12%-13%, between 13%-14%, between 14%-15%, between 15%-16%, between 16%-17%, between 17%-18%, between 18%-19%, between 19%-20%, between 20%-21%, between 21%-22%, between 22%-23%, between 23%-24%, between 24%-25%, between 25%-26%, between 26%-27%, between 27%-28%, between 28%-29%, or between 29%-30%.

In some embodiments, the fiber is contacted with the cross-linking agent. In some embodiments, the fiber is contacted with the cross-linking agent for about 1 second, about 10 seconds, about 30 seconds, about 1 minute, about 10 minutes, about 20 minutes, about 30 minutes, about 40 minutes, about 50 minutes, about 1 hour, about 2 hours, about 3 hours, about 4 hours, about 8 hours, about 12 hours, about 24 hours, about 48 hours, or about 72 hours.

In some embodiments, the fiber is contacted with the cross-linking agent for 1 second, 10 seconds, 30 seconds, 1 minute, 10 minutes, 20 minutes, 30 minutes, 40 minutes, 50 minutes, 1 hour, 2 hours, 3 hours, 4 hours, 8 hours, 12 hours, 24 hours, 48 hours, or 72 hours.

In some embodiments, the fiber is contacted with the cross-linking agent for from about 1 second to from about 10 seconds, from about 10 seconds to from about 30 seconds, from about 30 seconds to from about 1 minute, from about 1 minute to from about 10 minute, from about 10 minutes to from about 20 minutes, from about 20 minutes to from about 30 minutes, from about 30 minutes to from about 40 minutes, from about 40 minutes to from about 50 minutes, from about 50 minutes to from about 1 hour, from about 1 hour to from about 2 hours, from about 2 hours to from about 3 hours, from about 3 hours to from about 4 hours, from about 4 hours to from about 8 hours, from about 8 hours to from about 12 hours, from about 12 hours to from about 24 hours, from about 24 hours to from about 48 hours, or from about 48 hours to about 72 hours.

In some embodiments, the fiber is contacted with the cross-linking agent for between 1 second to 10 seconds, between 10 seconds to 30 seconds, between 30 seconds to 1 minute, between 1 minute to 10 minutes, between 10 minutes to 20 minutes, between 20 minutes to 30 minutes, between 30 minutes to 40 minutes, between 40 minutes to 50 minutes, between 50 minutes to 1 hour, between 1 hour to 2 hours, between 2 hours to 3 hours, between 3 hours to 4 hours, between 4 hours to 8 hours, between 8 hours to 12 hours, between 12 hours to 24 hours, between 24 hours to 48 hours, or between 48 hours to 72 hours.

In some embodiments, the cross-linking agent comprises a pH of about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, about 12.0, about 12.1, about 12.2, about 12.3, about 12.4, about 12.5, about 12.6, about 12.7, about 12.8, about 12.9, or about 13.0.

In some embodiments, the cross-linking agent comprises a pH of at least, at most or exactly 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, or 13.0.

### Fixing Agent

In some embodiments, the fiber is contacted with a fixing agent. In some embodiments, the fixing agent comprises a mordant. In some embodiments, the fixing comprises a metal salt. In some embodiments, the fixing agent comprises nickel sulphate.

In some embodiments, the fixing agent is present at a concentration of about 0.0001%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%.

In some embodiments, the fixing agent is present at a concentration of at least, at most or exactly 0.0001%, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30%.

In some embodiments, the fixing agent is present at a concentration of from 0.0001% to about 0.001%, about 0.001% to about 0.01%, about 0.01% to about 0.1%, about 0.1% to about 0.1%, about 0.1% to about 0.2%, about 0.2% to about 0.3%, about 0.3% to about 0.4%, about 0.4% to about 0.5%, about 0.5% to about 0.6%, about 0.6% to about 0.7%, about 0.7% to about 0.8%, about 0.8% to about 0.9%, about 0.9% to about 1%, about 1% to about 2%, about 2% to about 3%, about 3% to about 4%, about 4% to about 5%, about 5% to about 6%, about 6% to about 7%, about 7% to about 8%, about 8% to about 9%, about 9% to about 10%, about 10% to about 11%, 11% to about 12%, about 12% to about 13%, about 13% to about 14%, about 14% to about 15%, about 15% to about 16%, about 16% to about 17%, about 17% to about 18%, about 18% to about 19%, about 19% to about 20%, about 20% to about 21%, about 21% to about 22%, about 22% to about 23%, about 23% to about 24%, about 24% to about 25%, about 25% to about 26%, about 26% to about 27%, about 27% to about 28%, about 28% to about 29%, or about 29% to about 30%.

In some embodiments, the fixing agent is present at a concentration of between 0.0001%-0.001%, between 0.001%-0.01%, between 0.01%-0.1%, between 0.1%-0.1%, between 0.1%-0.2%, between 0.2%-0.3%, between 0.3%-0.4%, between 0.4%-0.5%, between 0.5%-0.6%, between 0.6%-0.7%, between 0.7%-0.8%, between 0.8%-0.9%, between 0.9%-1%, between 1%-2%, between 2%-3%, between 3%-4%, between 4%-5%, between 5%-6%, between 6%-7%, between 7%-8%, between 8%-9%, between 9%-10%, between 10%-11%, between 11%-12%, between 12%-13%, between 13%-14%, between 14%-15%, between 15%-16%, between 16%-17%, between 17%-18%, between 18%-19%, between 19%-20%, between 20%-21%, between 21%-22%, between 22%-23%, between 23%-24%, between 24%-25%, between 25%-26%, between 26%-27%, between 27%-28%, between 28%-29%, or between 29%-30%.

In some embodiments, the fiber is contacted with the fixing agent. In some embodiments, the fiber is contacted with the fixing agent for about 1 second, about 10 seconds, about 30 seconds, about 1 minute, about 10 minutes, about 20 minutes, about 30 minutes, about 40 minutes, about 50 minutes, about 1 hour, about 2 hours, about 3 hours, about 4 hours, about 8 hours, about 12 hours, about 24 hours, about 48 hours, or about 72 hours.

In some embodiments, the fiber is contacted with the fixing agent for 1 second, 10 seconds, 30 seconds, 1 minute, 10 minutes, 20 minutes, 30 minutes, 40 minutes, 50 minutes, 1 hour, 2 hours, 3 hours, 4 hours, 8 hours, 12 hours, 24 hours, 48 hours, or 72 hours.

In some embodiments, the fiber is contacted with the fixing agent for from about 1 second to from about 10 seconds, from about 10 seconds to from about 30 seconds, from about 30 seconds to from about 1 minute, from about 1 minute to from about 10 minute, from about 10 minutes to from about 20 minutes, from about 20 minutes to from about 30 minutes, from about 30 minutes to from about 40 minutes, from about 40 minutes to from about 50 minutes, from about 50 minutes to from about 1 hour, from about 1 hour to from about 2 hours, from about 2 hours to from about 3 hours, from about 3 hours to from about 4 hours, from about 4 hours to from about 8 hours, from about 8 hours to from about 12 hours, from about 12 hours to from about 24 hours, from about 24 hours to from about 48 hours, or from about 48 hours to about 72 hours.

In some embodiments, the fiber is contacted with the fixing agent for between 1 second to 10 seconds, between 10 seconds to 30 seconds, between 30 seconds to 1 minute, between 1 minute to 10 minutes, between 10 minutes to 20 minutes, between 20 minutes to 30 minutes, between 30 minutes to 40 minutes, between 40 minutes to 50 minutes, between 50 minutes to 1 hour, between 1 hour to 2 hours, between 2 hours to 3 hours, between 3 hours to 4 hours, between 4 hours to 8 hours, between 8 hours to 12 hours, between 12 hours to 24 hours, between 24 hours to 48 hours, or between 48 hours to 72 hours.

In some embodiments, the fixing agent comprises a pH of about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, about 12.0, about 12.1, about 12.2, about 12.3, about 12.4, about 12.5, about 12.6, about 12.7, about 12.8, about 12.9, or about 13.0.

In some embodiments, the fixing agent comprises a pH of at least, at most or exactly 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, or 13.0.

### Dyeing Agent

In some embodiments, the fiber is contacted with a dying agent. In some embodiments, the dyeing agent comprises a protein bonded light harvesting chromophore. In some embodiments, the dyeing agent comprises a commercial protein bonded light harvesting chromophore. In some embodiments, the dyeing agent comprises a lab-produced protein bonded light harvesting chromophore.

In some embodiments, the dyeing agent is present at a concentration of about 0.0001%, about 0.001%, about 0.0025%, about 0.005%, about 0.0075%, about 0.01%, about 0.0125%, about 0.015%, about 0.0175%, about 0.1%, about 0.125%, about 0.15%, about 0.175%, about 0.2%, about 0.225%, about 0.25%, about 0.275%, about 0.3%, about 0.325%, about 0.35%, about 0.375%, about 0.4%, about 0.425%, about 0.45%, about 0.475%, about 0.5%, about 0.525%, about 0.55%, about 0.575%, about 0.6%, about 0.625%, about 0.65%, about 0.675%, about 0.7%, about 0.725%, about 0.75%, about 0.775%, about 0.8%, about 0.825%, about 0.85%, about 0.875%, about 0.9%, about 0.925%, about 0.95%, about 0.975%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%. The concentration can be w/v or v/v. In one embodiment, the concentration is w/v.

In some embodiments, the dyeing agent is present at a concentration of at least, at most or exactly 0.0001%, 0.001%, 0.0025%, 0.005%, 0.0075%, 0.01%, 0.0125%, 0.015%, 0.0175%, 0.1%, 0.125%, 0.15%, 0.175%, 0.2%, 0.225%, 0.25%, 0.275%, 0.3%, 0.325%, 0.35%, 0.375%, 0.4%, 0.425%, 0.45%, 0.475%, 0.5%, 0.525%, 0.55%, 0.575%, 0.6%, 0.625%, 0.65%, 0.675%, 0.7%, 0.725%, 0.75%, 0.775%, 0.8%, 0.825%, 0.85%, 0.875%, 0.9%, 0.925%, 0.95%, 0.975%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 21%, 22%, 23%, 24%, 25%, 26%, 27%, 28%, 29% or 30%.

In some embodiments, the dyeing agent is present at a concentration from about 0.0001% to about 0.001%, about 0.001% to about 0.01%, about 0.01% to about 0.1%, about 0.1% to about 0.15%, about 0.15% to about 0.2%, about 0.1% to about 0.2%, about 0.2% to about 0.25%, about 0.2% to about 0.3%, about 0.3% to about 0.35%, about 0.3% to about 0.4%, about 0.4% to about 0.45%, about 0.4% to about 0.5%, about 0.5% to about 0.55%, about 0.5% to about 0.6%, about 0.6% to about 0.65%, about 0.6% to about 0.7%, about 0.7% to about 0.75%, about 0.7% to about 0.8%, about 0.8% to about 0.85%, about 0.8% to about 0.9%, about 0.9% to about 0.95%, about 0.9% to about 1%, about 1% to about 2%, about 2% to about 3%, about 3% to about 4%, about 4% to about 5%, about 5% to about 6%, about 6% to about 7%, about 7% to about 8%, about 8% to about 9%, about 9% to about 10%, about 10% to about 11%, 11% to about 12%, about 12% to about 13%, about 13% to about 14%, about 14% to about 15%, about 15% to about 16%, about 16% to about 17%, about 17% to about 18%, about 18% to about 19%, about 19% to about 20%, about 20% to about 21%, about 21% to about 22%, about 22% to about 23%, about 23% to about 24%, about 24% to about 25%, about 25% to about 26%, about 26% to about 27%, about 27% to about 28%, about 28% to about 29%, or about 29% to about 30%. The concentration can be w/v or v/v. In one embodiment, the concentration is w/v.

In some embodiments, the dyeing agent is present at a concentration of between 0.0001%-0.001%, between 0.001%-0.01%, between 0.01%-0.1%, between 0.1%-0.1%, between 0.1%-0.2%, between 0.2%-0.3%, between 0.3%-0.4%, between 0.4%-0.5%, between 0.5%-0.6%, between 0.6%-0.7%, between 0.7%-0.8%, between 0.8%-0.9%, between 0.9%-1%, between 1%-2%, between 2%-3%, between 3%-4%, between 4%-5%, between 5%-6%, between 6%-7%, between 7%-8%, between 8%-9%, between 9%-10%, between 10%-11%, between 11%-12%, between 12%-13%, between 13%-14%, between 14%-15%, between 15%-16%, between 16%-17%, between 17%-18%, between 18%-19%, between 19%-20%, between 20%-21%, between 21%-22%, between 22%-23%, between 23%-24%, between 24%-25%, between 25%-26%, between 26%-27%, between 27%-28%, between 28%-29%, or between 29%-30%. The concentration can be w/v or v/v. In one embodiment, the concentration is w/v.

In some embodiments, the fiber is contacted with the dyeing agent. In some embodiments, the fiber is contacted with the dyeing agent for about 1 second, about 10 seconds, about 30 seconds, about 1 minute, about 10 minutes, about 20 minutes, about 30 minutes, about 40 minutes, about 50 minutes, about 1 hour, about 2 hours, about 3 hours, about 4 hours, about 8 hours, about 12 hours, about 24 hours, about 48 hours, about 72 hours, about 96 hours, about 120 hours, or about 144 hours.

In some embodiments, the fiber is contacted with the dyeing agent for 1 second, 10 seconds, 30 seconds, 1 minute, 10 minutes, 20 minutes, 30 minutes, 40 minutes, 50 minutes, 1 hour, 2 hours, 3 hours, 4 hours, 8 hours, 12 hours, 24 hours, 48 hours, 72 hours, 96 hours, 120 hours or 144 hours.

In some embodiments, the fiber is contacted with the dyeing agent for from about 1 second to from about 10 seconds, from about 10 seconds to from about 30 seconds, from about 30 seconds to from about 1 minute, from about 1 minute to from about 10 minute, from about 10 minutes to from about 20 minutes, from about 20 minutes to from about 30 minutes, from about 30 minutes to from about 40 minutes, from about 40 minutes to from about 50 minutes, from about 50 minutes to from about 1 hour, from about 1 hour to from about 2 hours, from about 2 hours to from about 3 hours, from about 3 hours to from about 4 hours, from about 4 hours to from about 8 hours, from about 8 hours to from about 12 hours, from about 12 hours to from about 24 hours, from about 24 hours to from about 48 hours, from about 48 hours to about 72 hours, from about 72 hours to about 96 hours, from about 96 hours to about 120 hours, or from about 120 hours to about 144 hours.

In some embodiments, the fiber is contacted with the dyeing agent for between 1 second to 10 seconds, between 10 seconds to 30 seconds, between 30 seconds to 1 minute, between 1 minute to 10 minutes, between 10 minutes to 20 minutes, between 20 minutes to 30 minutes, between 30 minutes to 40 minutes, between 40 minutes to 50 minutes, between 50 minutes to 1 hour, between 1 hour to 2 hours, between 2 hours to 3 hours, between 3 hours to 4 hours, between 4 hours to 8 hours, between 8 hours to 12 hours, between 12 hours to 24 hours, between 24 hours to 48 hours, between 48 hours to 72 hours, between 72 hours to 96 hours, between 96 hours to 120 hours or between 120 hours to 144 hours.

In some embodiments, the dyeing agent comprises a pH of about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, about 12.0, about 12.1, about 12.2, about 12.3, about 12.4, about 12.5, about 12.6, about 12.7, about 12.8, about 12.9, or about 13.0.

In some embodiments, the dyeing agent comprises a pH of at least, at most or exactly 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, or 13.0.

### Temperature

In some embodiments, the methods of the disclosure are conducted at ambient temperatures. In some embodiments, an ambient temperature is about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C.

In some embodiments, an ambient temperature is 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C. or 50° C.

In some embodiments, an ambient temperature is about 10° C. to 11° C., from about 11° C. to about 12° C., from about 12° C. to about 13° C., from about 13° C. to about 14° C., from about 14° C. to about 15° C., from about 15° C. to about 16° C., from about 16° C. to about 17° C., from about 17° C. to about 18° C., from about 18° C. to about 19° C., from about 19° C. to about 20° C., from about 20° C. to about 21° C., from about 21° C. to about 22° C., from about 22° C. to about 23° C., from about 23° C. to about 24° C., from about 24° C. to about 25° C., from about 25° C. to about 26° C., from about 26° C. to about 27° C., from about 27° C. to about 28° C., from about 28° C. to about 29° C., from about 29° C. to about 30° C., from about 30° C. to about 31° C., from about 31° C. to about 32° C., from about 32° C. to about 33° C., from about 33° C. to about 34° C., from about 34° C. to about 35° C., from about 35° C. to about 36° C., from about 36° C. to about 37° C., from about 37° C. to about 38° C., from about 38° C. to about 39° C., from about 39° C. to about 40° C., from about 40° C. to about 41° C., from about 41° C. to about 42° C., from about 42° C. to about 43° C., from about 43° C. to about 44° C., from about 44° C. to about 45° C., from about 45° C. to about 46° C., from about 46° C. to about 47° C., from about 47° C. to about 48° C., from about 48° C. to about 49° C., or from about 49° C. to about 50° C.

In some embodiments, an ambient temperature is between 10° C.-11° C., between 11° C.-12° C., between 12° C.-13° C., between 13° C.-14° C., between 14° C.-15° C., between 15° C.-16° C., between 16° C.-17° C., between 17° C.-18° C., between 18° C.-19° C., between 19° C.-20° C., between 20° C.-21° C., between 21° C.-22° C., between 22° C.-23° C., between 23° C.-24° C., between 24° C.-25° C., between 25° C.-26° C., between 26° C.-27° C., between 27° C.-28° C., between 28° C.-29° C., between 29° C.-30° C., between 30° C.-31° C., between 31° C.-32° C., between 32° C.-33° C., between 33° C.-34° C., between 34° C.-35° C., between 35° C.-36° C., between 36° C.-37° C., between 37° C.-38° C., between 38° C.-39° C., between 39° C.-40° C., between 40° C.-41° C., between 41° C.-42° C., between 42° C.-43° C., between 43° C.-44° C., between 44° C.-45° C., between 45° C.-46° C., between 46° C.-47° C., between 47° C.-48° C., between 48° C.-49° C., or between 49° C.-50° C.

### Hunter L Scale

In some embodiments, the methods of the disclosure stabilizes the color of the dyed fiber as measured on the Hunter-L scale. In some embodiments, the Hunter-L scale does not fluctuate more than 20 units, more than 19 units, more than 18 units, more than 17 units, more than 16 units, more than 15 units, more than 14 units, more than 13 units, more than 12 units, more than 11 units, more than 10 units, more than 9 units, more than 8 units, more than 7 units, more than 6 units, more than 5 units, more than 4 units, more than 3 units, more than 2 units, or more than 1 unit after washing in water.

In some embodiments, the Hunter-L scale does not fluctuate from more than about 1 unit to 2 units, from more than about 2 units to 3 units, from more than about 3 units to 4 units, from more than about 4 units to 5 units, from more than about 5 units to 6 units, from more than about 6 units to 7 units, from more than about 7 units to 8 units, from more than about 8 units to 9 units, from more than about 9 units to 10 units, from more than about 11 units to 12 units, from more than about 12 units to 13 units, from more than about 13 units to 14 units, from more than about 14 units to 15 units, from more than about 15 units to 16 units, from more than about 16 units to 17 units, from more than about 17 units to 18 units, from more than about 18 units to 19 units, or from more than about 19 units to 20 units after washing in water.

In some embodiments, the method results in an obtained dyed fiber with a score of less than 70, less than 69, less than 68, less than 67, less than 66, less than 65, less than 64, less than 63, less than 62, less than 61, less than 60, less than 59, less than 58, less than 57, less than 56, less than 55, less than 54, less than 53, less than 52, less than 51, less than 50, less than 49, less than 48, less than 47, less than 46, less than 45, less than 44, less than 43, less than 42, less than 41, less than 40, less than 39, less than 38, less than 37, less than 36, less than 35, less than 34, less than 33, less than 32, less than 31, or less than 30 as measured on the Hunter-L scale.

pH

In some embodiments, the method is conducted at various pH. In some embodiments, the fabric is soaked in a composition at about pH 2, about pH 3, about pH 4, about pH 5, about pH 6, about pH 7, about pH 8, about pH 9, about pH 10, about pH 11, or about pH 12.

In some embodiments, the fabric is soaked in a composition at pH 2, pH 3, pH 4, pH 5, pH 6, pH 7, pH 8, pH 9, pH 10, pH 11 or pH 12.

In some embodiments, the method is conducted at various pH. In some embodiments, the fabric is soaked in a composition from about pH 2 to about pH 3, from about pH 3 to about pH 4, from about pH 4 to about pH 5, from about pH 5 to about pH 6, from about pH 6 to about pH 7, from about pH 7 to about pH 8, from about pH 8 to about pH 9, from about pH 9 to about pH 10, from about pH 10 to about pH 11, or from about pH 11 to about pH 12.

In some embodiments, the fabric is soaked in a composition between pH 2-pH 3, between pH 3-pH 4, between pH 4-pH 5, between pH 5-pH 6, between pH 6-pH 7, between pH 7-pH 8, between pH 8-pH 9, between pH 9-pH 10, between pH 10-pH 11, or between pH 11-pH 12.

### Protein Bonded Light Harvesting Chromophores

Phycobiliproteins are highly fluorescent water-soluble proteins that strongly absorb visible light with high extinction coefficients, high fluorescence quantum energy and minimal fluorescence quenching properties.

The light harvesting pigment-proteins described herein are all proteins with covalently bound bilin (linear tetrapyrrole) prosthetic groups, typified by those found in phycobiliproteins. All are water-soluble chromoproteins present in microalgae belonging to eukaryotic Rhodophyceae (red algae), Cryptophyceae, Glaucophyceae lineages as well as the Cyanophyceae (cyanobacteria, a.k.a. blue-green algae).

According to their light absorption and types of bilins, phycobiliproteins are commonly divided into four subclasses:
phycoerythrin (PE, pink-purple, λ max=540-570 nm);
phycocyanin's (PC, blue, λ max=610-620 nm);
phycoerythrocyanins (PEC, orange, λ max=560-600 nm); and,
allophycocyanins (APC, bluish-green, λ max=650-655 nm).

Phycobiliproteins assemble to form supramolecular complexes called phycobilisomes contributing to light harvesting and energy transfer processes. The pigments serve as solar energy collectors in the blue and green spectral region not absorbed by chlorophyll a. Phycobiliproteins are generally formed of two chromophore-linked subunits, a and B, and found in trimeric (αβ)*3 (MW~120 kDa) or hexameric units (αβ)*6 (~240 k Da). Phycoerythrin PE has been proposed by some authors to be used as a natural food red colorant, by analogy to the use of phycocyanin, a well-recognized natural blue colorant for food applications.

Phytochromes and their distant cyanobacterial relatives, the cyanobacteriochromes (CBCRs), utilize the same bilin pigments found in phycobiliproteins. When covalently bound to phytochromes and CBCRs, the bilin pigments become photoactive switches that are able to interconvert between two isomeric states with different absorption properties, unlike phycobiliproteins which are highly fluorescent. Each phytochrome and CBCR protein tunes the spectrum of the bound pigment to respond to different wavelengths of light, and indeed some are even fluorescent like phycobiliproteins. Hence, the range of CBCR and phytochrome color is quite broad. Moreover, most CBCRs and phytochromes are capable of converting between different colored states due to the unique protein environment of their bound bilin pigment.

In some embodiments, the disclosure utilizes CBCR-6012, and variants thereof, which is derived from a larger protein found in the cyanobacterium Nostoc punctiforme. CBCR-6012 is a small biliprotein with a covalently attached bilin phycocyanobilin (PCB)—the same pigment that is bound to spirulina. CBCR-6012 can be synthesized by fermentation in a bacterial strain that expresses the structural ‘apoprotein-encoding’ gene and the genes for synthesis of PCB from endogenous heme (Gambetta, G. A. and J. C. Lagarias (2001) PNAS 98 (19): 10566-10571; Rockwell, N. C., S. S. Martin and J. C. Lagarias (2012) Biochemistry 51 (48): 9667-9677).

CBCR-6012 is a blue-colored protein when isolated from dark-grown E. coli cultures. Under red light, the protein converts to a red colored protein that can be converted back to the blue state under green light. Under white light, the protein becomes purple due to a mixture of the two colored states that are produced. CBCR-6012 (and other CBCRs) can be produced in large quantities by fermentation. CBCR-6012 can be used in similar colorant applications known for phycobiliproteins such as spirulina; however, CBCR-6012 can adopt two colorant states. Therefore, CBCR-6012 may provide an additional benefit as a colorant.

In some embodiments, the protein bonded light harvesting chromophore comprises a phycoerythrin.

In some embodiments, the protein bonded light harvesting chromophore comprises a phycocyanin.

In some embodiments, the protein bonded light harvesting chromophore comprises a phycoerythrocyanin.

In some embodiments, the protein bonded light harvesting chromophore comprises an allophycocyanin.

In some embodiments, the protein bonded light harvesting chromophore comprises a tetra pyrrole ring.

In some embodiments, the protein bonded light harvesting chromophore comprises a modified tetra pyrrole ring.

In some embodiments, the protein bonded light harvesting chromophore comprises a modified tetra pyrrole ring, wherein the tetra pyrrole ring has a different light harvest capacity, as compared to an unmodified tetra pyrrole ring.

In some embodiments, the protein bonded light harvesting chromophore comprises a phycobiliprotein.

In some embodiments, the protein bonded light harvesting chromophore is produced by a green or blue algae.

In some embodiments, the protein bonded light harvesting chromophore is produced by a Spirulina sp.

In some embodiments, the protein bonded light harvesting chromophore comprises a cyanobacteriochrome.

In some embodiments, the protein bonded light harvesting chromophore is produced by a Cyanobacteria.

### Therapeutic Uses

Blue colored pigment-proteins have several health-promoting activities including their potent antioxidant, antibacterial, anti-inflammatory, and immune modulator activities and they are natural pigments in some food products; for example, aqueous extract of non-purified spirulina has been added in dairy products, ice creams, yogurts, isotonic beverages, confectionary, and jellies (See Foods 2020, 9, 244). The vibrant blue color from spirulina is preferred over other less-bright natural colorants in confectionary products. The red-colored phycobiliprotein, phycoerythrin (PE) has been mostly used as a fluorescent probe in biomedical studies.

The benefits of spirulina protein-bonded pigments in skin care applications are well documented (See Cosmetics 2021, 8, 7). The most common of cosmetic actions, supported by the literature and claimed by the market, are the anti-acne properties in moisturizing, antioxidant, and skin brightening properties, and wound healing properties. US Patent application 2020/0155642A1 discloses the purified extract of pigment-protein (phycobiliproteins) in oral, topical, mucosal, ophthalmological applications.

In some cases, a fiber dyed using the methods and/or compositions provided herein can provide a therapeutic property to a textile, fabric, yarn or garment comprising said dyed fiber. In some cases, the therapeutic properties can be relayed to an individual wearing the textile, fabric, yarn or garment comprising the fiber dyed using a method and/or composition provided herein. The therapeutic property can comprise wound-healing properties, anti-inflammatory properties, antioxidant properties, antibacterial properties, immune modulatory properties or any combination thereof.

### EXAMPLES

The present disclosure is further illustrated by reference to the following Examples. However, it should be noted that these Examples, like the embodiments described above, are illustrative and are not to be construed as restricting the scope of the invention in any way.

### Example 1: Description of Textile Dyeing Process

### Purpose

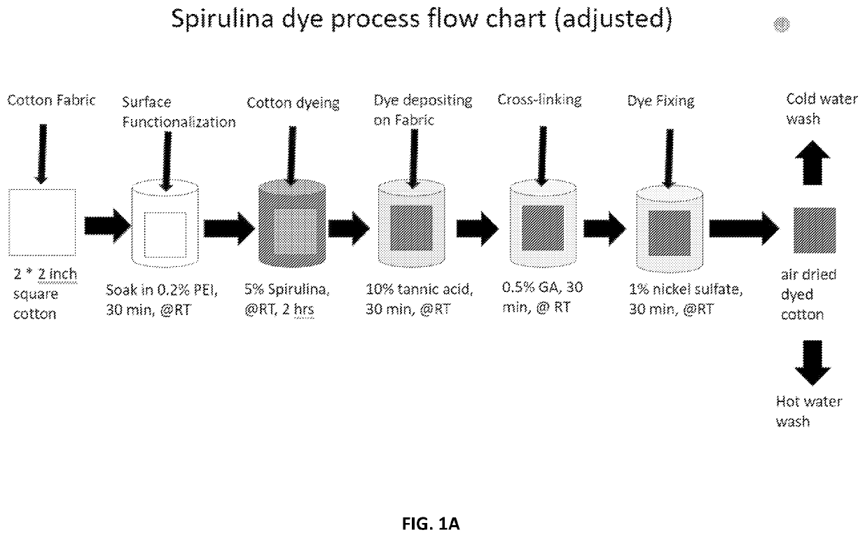

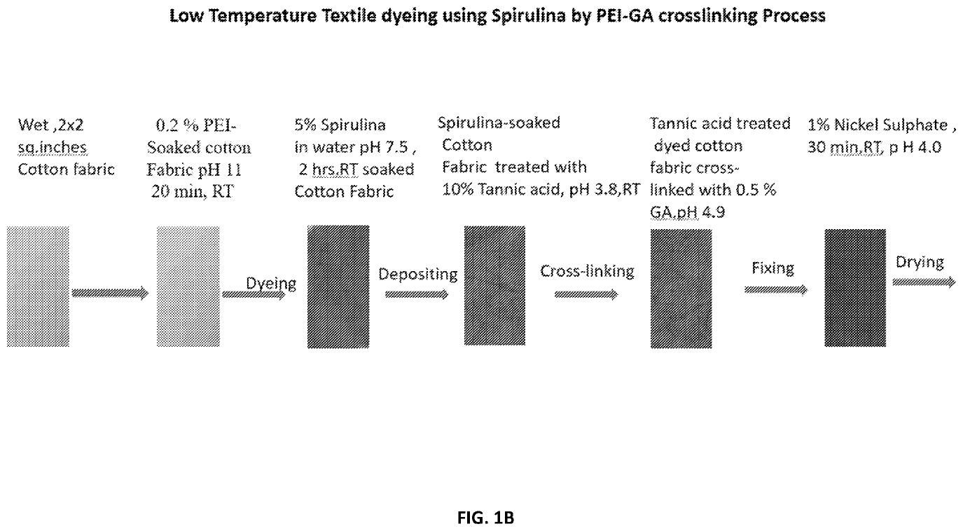

The purpose of this example was to provide a detailed description of the textile dyeing process of the present disclosure used in subsequent examples. A schematic of this process is shown in FIG. 1A.

### Methods

Both commercial spirulina and lab-produced light harvesting chromophores, such as CBCR-6012, may be used in the methods of the current disclosure.

Protein bonded light harvesting chromophore-CBCR-6012 Untagged: Transformed colonies were used to inoculate 6×100 ml LB cultures containing Kanamycin-20 (20 g/mL) and Chloramphenicol-20 (20 μg/mL). These were the starter cultures for the 6×1 liter cell growth for protein expression. The starter cultures were grown overnight at 37° C. degrees, with shaking at 200 RPM. The next day, 6×1 liter LB cultures in Fernbach flasks, each containing Kan 20 and Cam 20 plus 84 mg of ALA (Frontier Scientific), were inoculated with 5 ml of starter culture and grown to at 37° C., with shaking at 200 RPM, in a dark shaker until OD 600=0.8. This process took approximately 3-4 hours. Protein expression was induced with 1 ml of 1 M IPTG (Teknova) per flask. The temperature was then reduced to 20° C. and shaking was continued overnight at 120 RPM.

Upon harvesting, the culture was spun at 5,000×G RPM for 10 minutes to isolate cells. The cell pellet was scraped into a 50 mL conical tube and resuspended in 60 ml Buffer A (20 mM HEPES/10% glycerol pH 7.8). The cells were next lysed via physical disruption using a microfluidizer until the solution changed from milky to homogeneous and translucent. High speed centrifugation (35,000×G) was utilized to isolate the colored supernatant from the cell debris. This colored supernatant was purified using chitin resin (NEB) using the manufacturer's instructions with previously published modifications, followed with a final dialysis against TKKG buffer (25 mM TES-KOH (pH 7.8), 100 mM KCl, and 10% (v/v) glycerol). (See Biochemistry 2012, 51, 48, 9667-9677).

Commercial Spirulina, a vibrant blue color Spirulina powder, was purchased from Lone Star Botanicals (P.O Box 6910 Tyler, TX 75711). An aqueous solution of spirulina powder was made by dissolving 125 mg in 99.9 milliliters of water (0.125% w/v). The pH was adjusted to pH 7.0 using dilute 5% sodium hydroxide. The purity index (PI) ratio was calculated by measuring the optical density of the spirulina solution at 650 nm and 280 nm in a spectrophotometer (Molecular Devices, Spectromax M3 3860 N, First St, San Jose, CA 95134). The PI ratio of the Spirulina sample was 2.12 and the PI ratio of untagged CBCR-6012 was 0.36.

Protein determination was performed using Coomassie protein assay reagent obtained from Thermo Scientific (168 Third Avenue, Waltham, MA, 02451). Standards were diluted in water following supplier recommendations. Spirulina and CBCR samples were diluted to be in the linear range of the standards and the readings were taken in Spectromax M3 Spectrometer at 595 nm.

Cotton fabric was wetted in water and then soaked in a 0.2% PEI solution, pH 11.0 (MW 10,000 branched from Polysciences Inc., 400 Valley Road, Warrington, PA 18976) for 30 minutes as shown in FIG. 1A-1B. The PEI-soaked cotton fabric was then transferred to a dyeing bath containing 5% spirulina in water (pH 6.0), for a final pH of 7.5 (Lone Star Botanicals Box 6910 Tyler, TX 75711) at room temperature for more than 2 hours. The spirulina dyed cotton was then soaked in an acidic mordant, such as 10% tannic acid, pH 3.8 (Bean Town Chemical, 9 Sagamore Park Road, Hudson, NH 03051) or citric acid, to deposit spirulina on the surface of the fabric for 30 minutes. Then, the spirulina was cross-linked with glutaraldehyde, GA (50% Glutaraldehyde from VWR Life science, Radnor Corporate Center Building One, Suite 200, 100 Matsonford Road Radnor, PA 19087) for 30 minutes as shown in FIGS. 1A-1B. Next, the cross-linked spirulina was fixed onto the fabric using nickel sulphate (Thermos Scientific, 168 Third Avenue, Waltham, MA 02451) as shown in FIGS. 1A-1B.

### Results and Conclusion

The pictures of the treated cotton fabric are illustrated in FIG. 1B. As can be seen, the method resulted in dying cotton fabric to a dark blue color. This example demonstrated that cotton fabric may be dyed to a dark blue color using methods of the disclosure.

### Example 2: Determine Effect of Soaking Time of Cotton Fabric in PEI on Dyed Fabric

### Purpose

The purpose of this experiment was to determine the effect of soaking time of cotton fabric in 0.2% Polyethyleneimine (PEI) solution on the darkness of spirulina dyed fabric.

### Methods

Six squares of cotton measuring two inches by two inches were cut and soaked in 2 milliliters of a 0.2% PEI solution (pH 11) for 30 minutes at room temperature. After soaking, the cotton fabric was squeezed to remove excess liquid and moved to a test tube containing 3 milliliters of 5% w/v spirulina solution in water (pH 6.0) for dyeing at room temperature. Samples were collected at different time intervals, i.e., 30 min, 1 hour, 2-hours, 3 hours, 8 hours, and 24 hours. After the appropriate dyeing time, the dyed cotton was squeeze dried and moved to soak in 10% tannic acid (pH 3.0) for 30 minutes at room temperature to allow the deposition of spirulina color onto the surface of the fabric. The deposited spirulina was then cross-linked by soaking in 0.5% glutaraldehyde, pH 4.5 (50% Glutaraldehyde from VWR Life science, Radnor Corporate, Center Building One, Suite 200,100 Matsonford, Radnor, PA 19087) for 30 min. The cross-linked spirulina was then fixed on the surface of cotton fabric by soaking the fabric in 1% Nickel sulphate (pH 4.5) for 30 minutes. The excess of the unbound color was removed from fabric by rinsing in water followed by air drying overnight before further analysis.

### Results and Conclusion

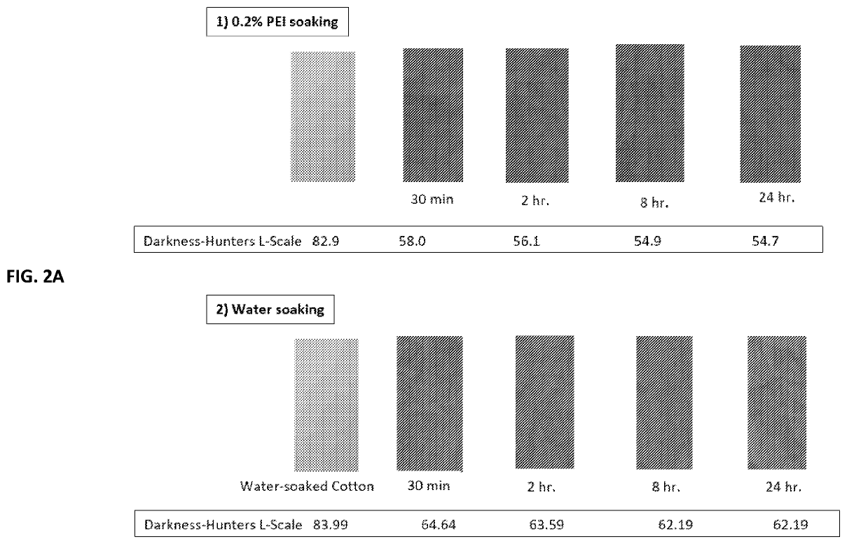

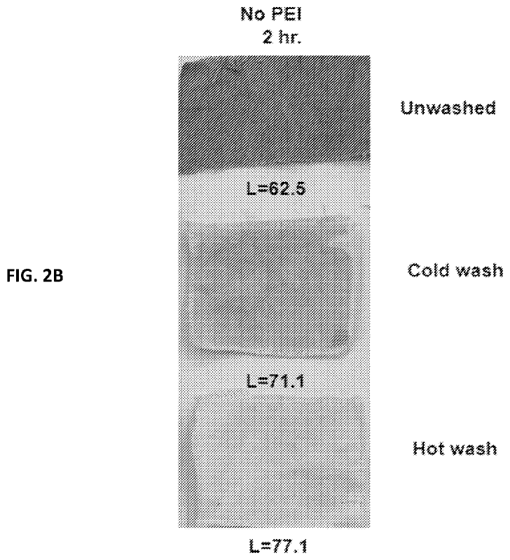

The above conditions were applied to cotton fabric soaked in water instead of 0.2% PEI for comparison (FIG. 2A). The darkness of the dry dyed cotton fabric was measured using portable Hunters' Colorimeter (FIG. 2A) from CTI (Shenzhen Wave Optoelectronics Technology Co, Ltd., 401 Building 40, Chuyun Road, Shenzhen, Guangdong, China). As it can be seen in FIG. 2A, the darkness of spirulina dyed fabric was increased (decreasing L scale in Hunters' colorimeter) with increasing soaking time in 5% spirulina until 8 hours and further increase in soaking time did not show a significant increase in the darkness of the fabric. Whereas, the darkness of the cotton fabric dyed with 5% spirulina, just soaked in water without PEI did not retain any darkness after washing with cold or hot water containing commercial detergent (FIG. 2B).

The results of this example showed that the surface functionalization of cotton fabric with a cationic polymer such as, for example, PEI resulted in adsorption of spirulina color to cotton surface.

### Example 3: Determine Effect of PEI Concentrations on Dyed Fabric

### Purpose

The purpose of this experiment was to determine the effect of various PEI concentrations, ranging from 0.02%, 0.2%, 2.0% and 5%, on the darkness of spirulina dyed fabric.

### Methods

After soaking fabric for 30 minutes in PEI with concentrations ranging from 0.02%, 0.2%, 2.0% and 5%, the PEI-soaked cotton fabrics were squeezed to remove the excess liquid and then transferred to separate tubes containing 5% spirulina aqueous solution (3 milliliters). The pH of solution in each tube was adjusted to pH 8.5 using dilute hydrochloric acid after 30 minutes and then continued soaking for additional 90 minutes. The dyeing process was continued with tannic acid, cross-linking with glutaraldehyde, and then treated with Nickel sulphate (1%), as described in Example 2. The dyed cotton fabrics were then rinsed twice with water and air dried overnight.

### Results and Conclusion

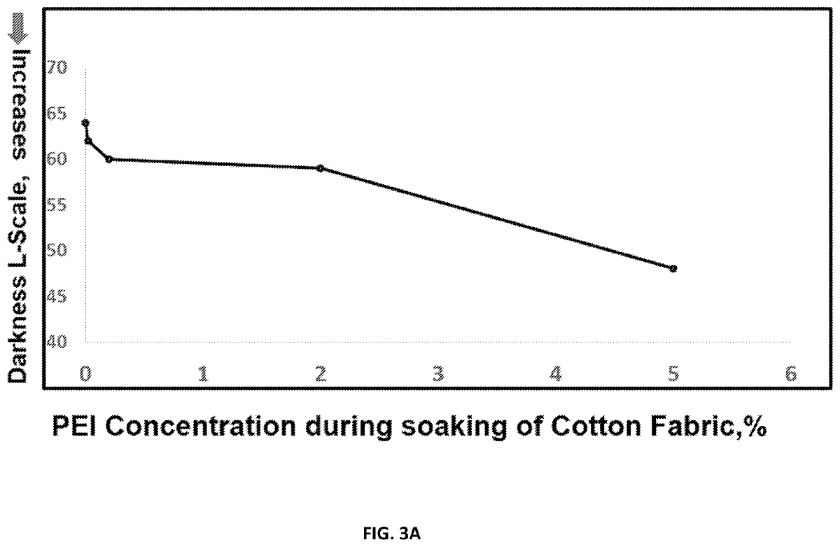

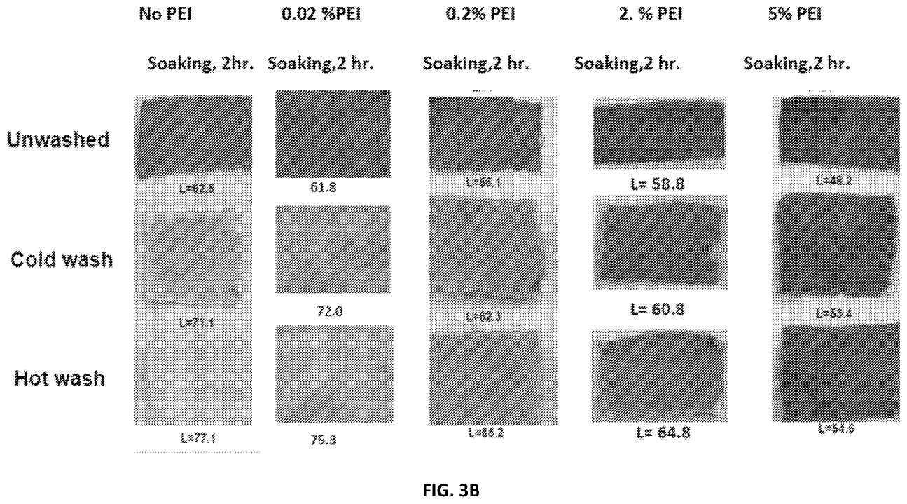

The measurement for darkness of the dried dyed cotton fabrics were measured by Hunters' colorimeter as described earlier in Example 2. These fabrics were then subjected to cold and hot wash and dried followed by measuring for the darkness. The results in FIG. 3A and FIG. 3B showed that the darkness of the spirulina dyed cotton fabric was increased in the darkness of the fabric with increasing concentrations of PEI during soaking of the cotton fabric (Table 1).

TABLE 1
Effect of PEI soaking concentration of cotton fabric
on the darkness of the spirulina dyed cotton fabric.

| | | |
|---|---|---|
| PEI | Darkness | Hunters' L-Scale |

| Concentration | Spirulina Dyed | Cold | Hot |
|---|---|---|---|
| (%) | Cotton Fabric | Wash | Wash |
| 0 | 63.8 | — | 82.2 |
| 0.02 | 61.8 | 72 | 75.3 |
| 0.2 | 59.5 | 64.4 | 67.5 |
| 2 | 58.8 | 60.5 | 64.8 |
| 5 | 48.2 | 66.5 | 54.6 |

Both cold and hot wash of the dyed fabric using conventional detergent showed a marked differences in the retention of the darkness of the dyed cotton fabric (Table 1). The darkness of both cold and hot wash of the dyed cotton fabric, was increased (decreasing in L number) with increasing concentrations of PEI during soaking of the cotton fabric.

The results of this experiment showed that surface functionalization of cotton fabric resulted in irreversible binding and adsorption of color.

### Example 4: Determine Effect of Multiple Dyeing of 0.2% PEI Treated Cotton Fabric

### Purpose

The purpose of this experiment was to determine the effect of multiple dyeing cycles using 0.2% PEI treated cotton fabric, as described in Example 2.

### Methods

Four 5-inch square pieces of cotton were cut and soaked in 350 milliliters of 0.2% PEI for 30 minutes at room temperature. The cotton was squeezed to remove excess solution and then moved to a 400-milliliter solution comprising 5% spirulina, then incubated for 2 hours at room temperature. The pH was adjusted to pH 8.5 using dilute Hydrochloric acid after 30 minutes. The cotton was then squeeze dried and moved to 10% solution of tannic acid, incubated for 1 hour followed by cross-linking of the deposited spirulina by soaking in 0.5% glutaraldehyde solution for 30 minutes. Then, the fabric was squeezed to remove the excess of solution and was left to dry overnight. The next day, a 2.5-inch square dyed fabric was cut from swatch and reserved for fixing with Nickel sulphate. The remaining swatches were dyed either a second, third, or fourth time by repeating the steps as described earlier and using the same solutions used for first dyeing: 0.2% PEI, 5% spirulina, 10% tannic acid and 0.5% Glutaraldehyde. Then, all the dyed samples (four samples) were soaked in 1% Nickel sulphate for fixing the color on to the fabric. The dyed fabric was air-dried overnight and again rinsed with water to remove unbound color to the fabric followed by air drying and measured the darkness of the color using Hunter-L darkness colorimeter.

### Results and Conclusion

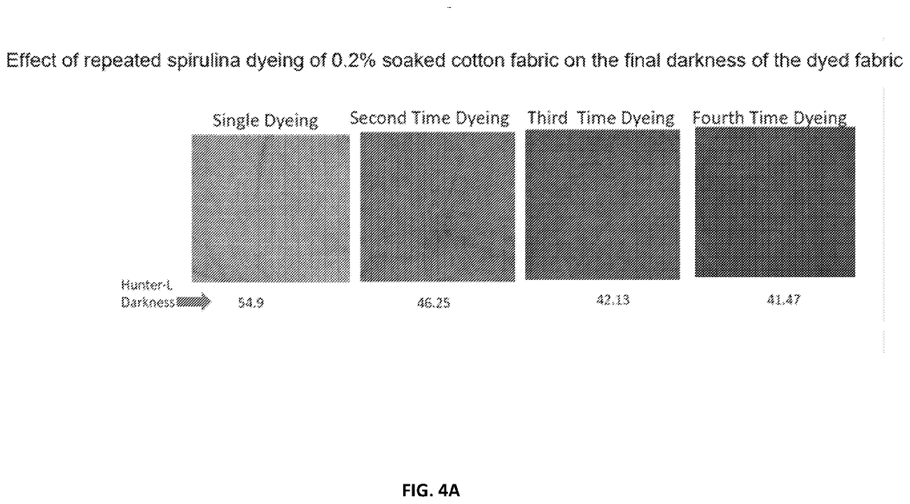

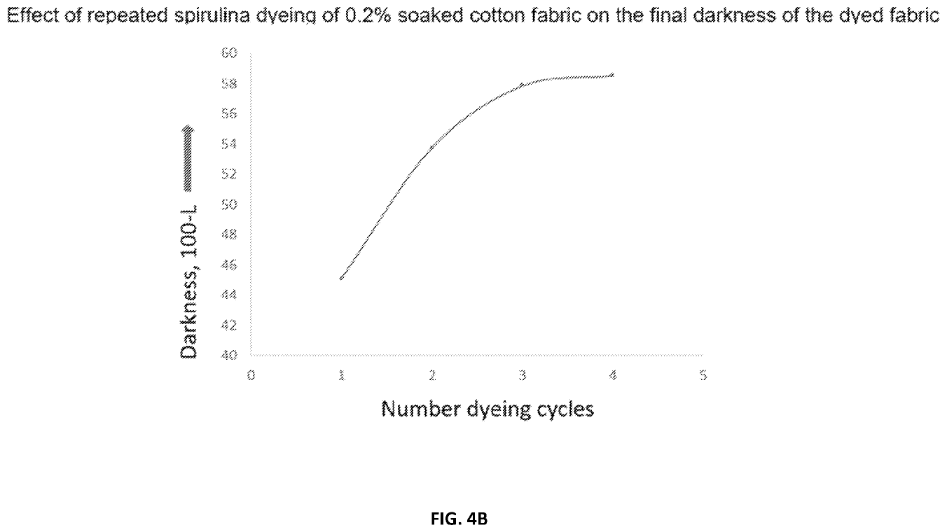

As can be seen in FIG. 4A and FIG. 4B, the Hunter-L darkness decreased with additional dyeing times. The greatest increase in darkness occurred during the first and second dyeing cycles, with smaller increases occurring after subsequent cycles. The data showed that the greatest increase in the darkness of the color occurred in the first and second dyeing cycle followed by a slight increase in the third dyeing cycle.

### Example 5: Determine Effect of Cold and Hot Washes on Dyed Fabric

### Purpose

The purpose of this experiment was to determine the effect a cold wash and a hot wash of the darkness of the dyed fabric.

### Methods

Both cold (25° C.) and hot (55° C.) washes of 5% PEI-soaked cotton fabric dyed with 5% spirulina were studied under a typical detergent wash condition. Dyed cotton fabric swatches were allowed to dry completely before subjecting to wash conditions in a portable washing machine. A small, portable washing machine from East Doll Company for washing small items was used (Zhongshan Donlim Weili Fusha Industrial Park, Fusha Town, Zhongshan City, Guangdong, China). For simulating cold wash, 3 milliliters of a commercial detergent product, liquid Tide®, were added to the portable washing machine with 1.5 liters of water (25° C.). Fabric was washed for 30 minutes, then the wash water was drained, and a fresh 1.5 liters water was added again to the washing machine for a 10-minute rinse cycle. Two rinses were carried out to complete the wash cycle. Washed fabric was left to air dry overnight to ensure complete drying before analysis.

For hot wash cycles, 1.5 liters of tap water were heated in an electric kettle up to 50° C. and then transferred to washing machine with milliliters of Tide®, with Oxiclean®. The cotton fabric was similarly washed for 30 minutes, followed by two 1-minute rinses with fresh tap water. The fabric was allowed to dry overnight. Both cold and hot single washed cotton fabric were again subjected to repeated wash cycle for 4 times, as described earlier.

### Results and Conclusion

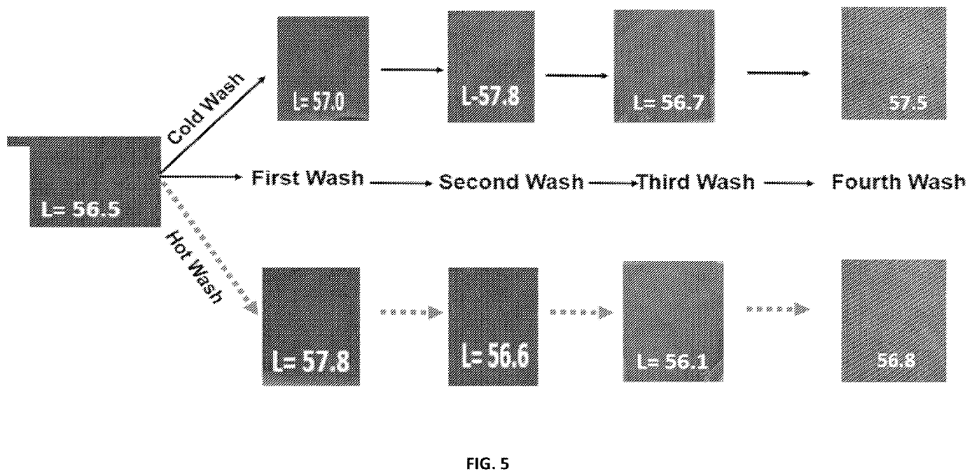

The color darkness of the dried fabric was determined using Hunters' colorimeter and the data were presented in FIG. 5. The results in FIG. 5 showed that the darkness of the dyed cotton fabric remained constant with repeated washings, either cold or hot wash indicating an irreversible binding of spirulina to 5% PEI treated cotton fabric.

The results of this experiment demonstrated that fabric dyed using methods of the current disclosure may be used washed in both cold and hot water without decreasing the darkness of the fabric.

### Example 6: Determine Effect of Glutaraldehyde Concentration on Fabric Darkness

### Purpose

The purpose of this example was to determine if glutaraldehyde concentration increases the darkness of a cotton fabric.

### Methods

The effect of glutaraldehyde concentration was studied on the darkness of spirulina dyed cotton fabric using 5% PEI-soaked cotton fabric followed by ambient temperature spirulina dyeing process. In a typical experiment, three pieces of cotton fabric, 2-inch square were taken and soaked together in 9 milliliters of 5% PEI solution for 30 minutes at room temperature. The PEI-soaked cotton fabrics were taken out from PEI soaking and squeezed to remove the excess of PEI solution from the fabric and moved to 9 milliliters of 5% spirulina solution containing tube. The pH of the solution was adjusted to pH 8.6 after 15 minutes and continued soaking for a total of 2 hours. The spirulina dyed cotton fabrics were then soaked in 9 milliliters of 10% tannic acid for 30 minutes. Three separate tubes containing 3 milliliters each of different concentrations of glutaraldehyde, i.e., 0.1%, 5.0% and 10% were taken and 10% tannic acid treated cotton fabric was transferred to GA containing tubes and soaked for 30 minutes followed by finishing the dyeing process by soaking the glutaraldehyde treated cotton fabric in 1% nickel sulphate for 30 minutes. The dyed cotton fabrics were then squeezed to remove excess of solution and rinsed with water twice before air drying at room temperature. A control sample was used in the dyeing process without glutaraldehyde treatment for comparison.

### Results and Conclusion

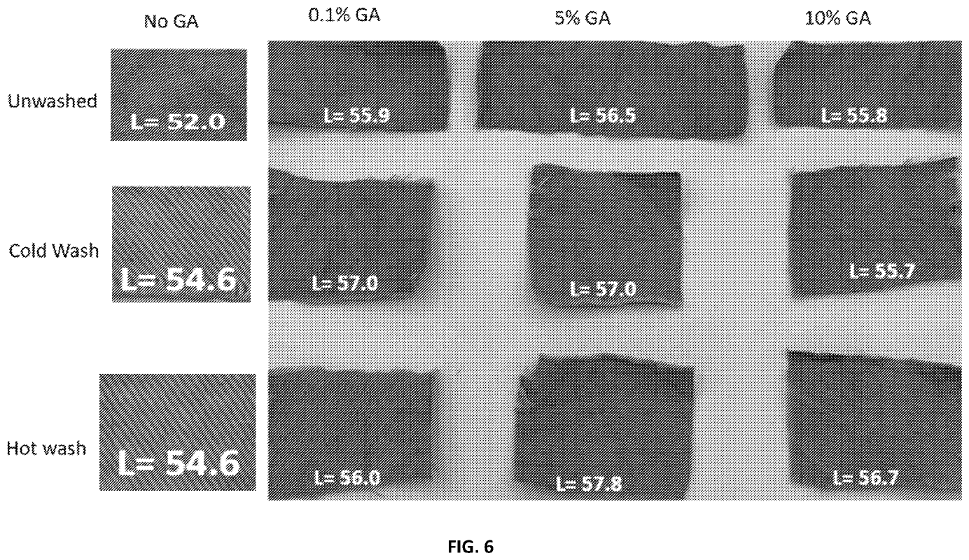

The results in FIG. 6 showed that the addition of each binding component causes an increasing shift of dyed cotton fabric darkness to a greener shade. The fabric with no PEI or GA was a light blue color. However, with the PEI and increasing concentrations, it started to show a slight greenish undertone and upon washing the swatches, a nice clean faded blue jean. The treatment of GA for cross-linking resulted in the color to shift even greener.

The results of this experiment demonstrated that the methods of the present disclosure allows for increased choice of color for dying fabric while maintaining color darkness.

### Example 7: Determine Effect of Textile Dyeing Process On Different Types Of Fabric

### Purpose

The purpose of this experiment was to determine if the textile dyeing processes of the present disclosure were effective on different types of fabrics.

### Methods

The ambient temperature textile dyeing process as described in FIG. 1A was used to dye the protein fabrics; i.e., silk, wool, jute, collagen, keratin, etc. In a typical experiment, two swatches of silk measuring 2.5-inch squares were cut and was soaked in 8 milliliters of 5% PEI solution at room temperature for 30 minutes. The PEI-soaked silk swatches were then squeezed to remove excess of PEI solution from the fabric and transferred to 8 milliliters solution of 5% spirulina solution. The pH of the spirulina solution was then adjusted from pH 10 to pH 8.9 using 5% phosphoric acid (100 μl). Swatches were then moved to 8 milliliters of 10% tannic acid for 30 minutes at room temperature. One of the tannic acids treated swatches was then soaked in 3 milliliters of 5% GA for 30 minutes. Both swatches with and without GA treatment were then treated with 1% nickel sulphate for fixing the color onto the protein fabric. The swatches were then air died overnight and appropriate measurements were made.

### Results and Conclusion

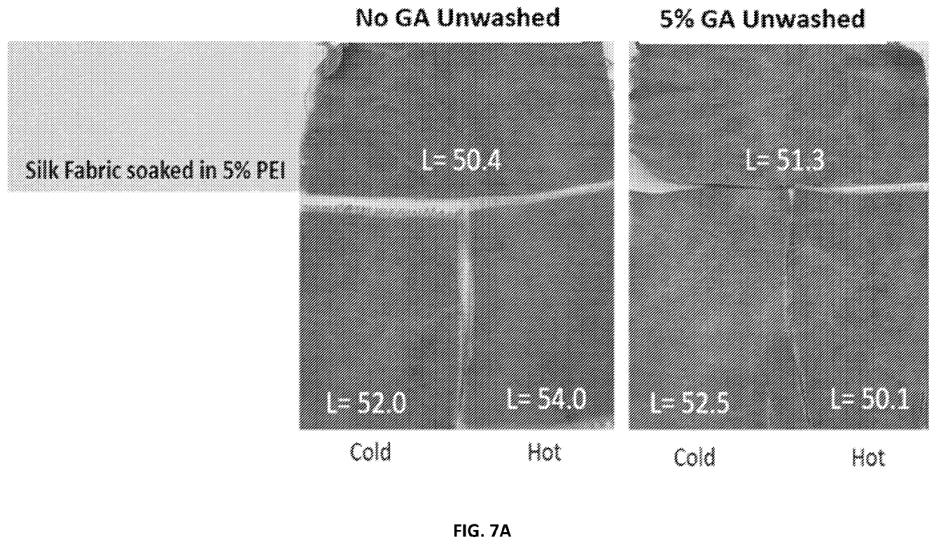

The results shown in FIG. 7A and FIG. 7B showed that the protein fabric dyed with spirulina using the process of the present disclosure did not lose the darkness of the fabric after cold or hot wash with commercial detergent product. Cross-linking using 5% GA after tannic acid treatment yielded a greenish hue to the dyed fabric (FIG. 7A and FIG. 7B). The results of this experiment demonstrated that cross-linking with GA can yield a fabric with a greenish hue without losing darkness of color after washing.

### Example 8: Determine Effect of Poly Cationic Natural Polymer on Surface Functionalization

### Purpose

The purpose of this example was to determine if a natural poly cationic polymer can be used for surface functionalization in the dyeing process of the current disclosure.

### Methods

Chitosan, a deacetylated chitin is the second largest carbon source naturally occurring biodegradable poly cat ionic natural polymer. Chitosan is positively charged by amino groups, making it suitable for binding to negatively charged molecules. The application of chitosan on wool fabric resulted in improvement on dyeability, shrink proofing and wettability. The effect of chitosan on the cotton fabric surface functionalization in the ambient temperature textile dyeing process of the present disclosure was studied. In a typical experiment, the pH of a 5% acetic acid solution was adjusted to pH 4.0 using sodium hydroxide. 2.5 gram of powdered chitosan, (Thermo Scientific Chemicals 168 Third Ave Waltham, MA 02451) was suspended in 10 milliliters of pH-4.0 adjusted acetic acid solution and mixed very well for mixing and solubilization. Then, a 2.5-inch square piece of cotton swatch was soaked in 2.5% chitosan solution for 30 minutes at room temperature. The chitosan-soaked cotton fabric was the removed and squeezed to remove the excess of chitosan solution and moved to 10 milliliters of 5% spirulina solution (pH 6.5). The cotton fabric was allowed to equilibrate in spirulina solution for 30 minutes. The pH of the spirulina solution dropped to pH 4.5 because of soaking of chitosan treated fabric. This caused the localized deposition of spirulina on to the surface of cotton fabric. This resulted in the elimination for the need of tannic acid treatment in the dyeing process. The spirulina dyed cotton fabric was cut into two pieces and one piece was treated with 5% GA followed by fixing with nickel sulphate (1% for 30 min) and the other piece was treated with nickel sulphate without GA treatment.

### Results and Conclusion

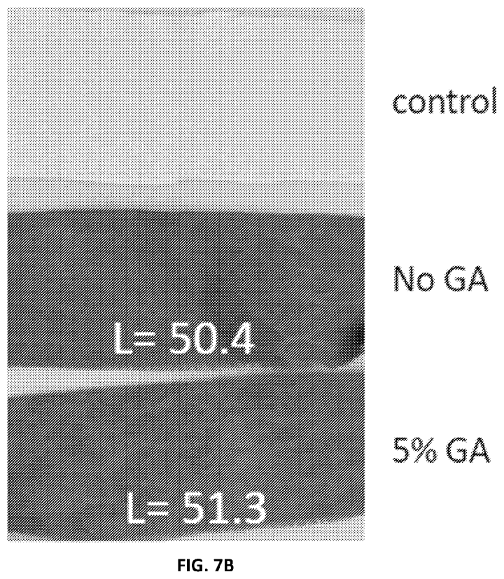

The results in FIG. 8 showed that chitosan, a natural poly cationic polymer, surface functionalization of the cotton fabric was similar to PEI on the spirulina dyeing process except there was no loss of darkness of the dyed fabric from cold or hot wash with commercial detergent product. The cotton fabric without GA treatment was bluer compared to GA treated cotton fabric (FIG. 8). This experiment demonstrated that the final darkness of the cotton fabric may be increased using either more concentrated chitosan solution or multiple dyeing.

### Example 9: Surface Functionalization Using Poly Cationic Polymer

### Purpose

The purpose of this experiment was to determine how surface functionalization using a poly cationic polymer affected the darkness of cotton fabric.

### Methods

A poly anionic polymer, i.e., polyacrylamide, Tramfloc®146 (146 TramFloc, Inc. 6046 FM 2920 Road #615, Spring, TX 77379) was studied in ambient temperature textile dyeing process of the present disclosure. Tramfloc® 146 was diluted using water to a final concentration of 0.625% (pH-6.3). Then 2.5-inch square piece of cotton swatch was soaked in 0.625% Tramfloc® 146 solution for 30 minutes at room temperature. The pH after soaking was pH 6.7. The soaked cotton was squeezed to remove the excess of solution and transferred to 8 milliliters of 5% aqueous solution of spirulina and soaked for 2 hours. Here again, the excess of spirulina solution was removed by squeezing the fabric and then soaked in 10% tannic acid for 30 minutes. The dyed cotton fabric was then cut into two pieces and one of the pieces moved straight to 1% nickel sulphate for final fixing the dye without GA treatment. The other piece was soaked in 5% GA for 30 minutes followed by treating with 1% nickel sulphate.

### Results and Conclusion

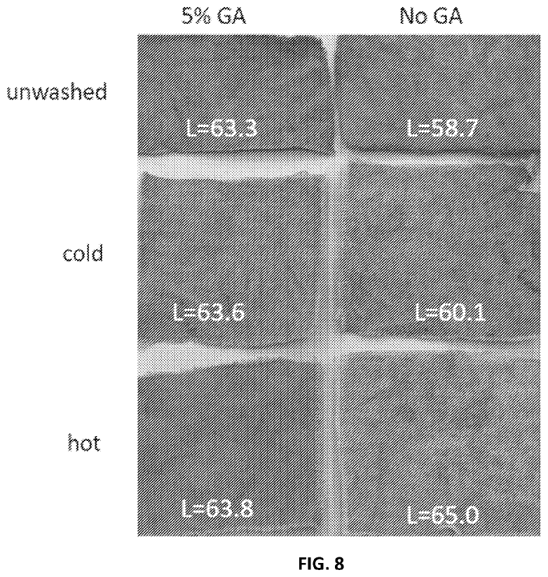

The darkness of the cotton fabrics after dyeing with spirulina with and without GA treatment is shown in FIG. 9. The darkness of the cotton fabric dyed with spirulina after surface functionalization using an anionic polymer, like polyacrylamide, was comparable to cotton fabric treated with poly cationic polymer (Hunter L-number of 53 vs 55, respectively). However, both cold and hot wash using commercial detergent product showed a significant loss of the color, but the loss of darkness was less with GA treated cotton fabric (FIG. 9).

The results of this experiment showed that treatment with GA decreases loss of color darkness after washing with a commercial detergent product.

### Example 10: Determine Effect of the Low Temperature Textile Dyeing Process for Synthetic Fabrics

### Purpose

The purpose of this experiment was to determine if the textile dyeing process of the present disclosure was effective on synthetic fabrics, i.e., polyester.

### Methods

The textile dyeing process described in FIG. 1A was used to dye polyester. In a typical experiment, two swatches of polyester, measuring 2.0-inch squares were cut and were soaked in 8 milliliters of 5% PEI solution at room temperature for 30 minutes. The PEI-soaked polyester was squeezed to remove the excess of solution and transferred to 8 milliliters of 5% aqueous solution of spirulina and soaked for 2 hours at room temperature. Here again, the excess of spirulina solution was removed by squeezing the polyester fabric and then soaked in 10% tannic acid for 30 minutes. The dyed polyester fabrics were then transferred to 5% GA solution and soaked for 30 minutes followed by washing with cold water to remove the unbound color. The dyed polyester fabrics were then air-dried at room temperature. The dried polyester swatches were then subjected to hot water wash as described in Example 5.

### Results and Conclusion

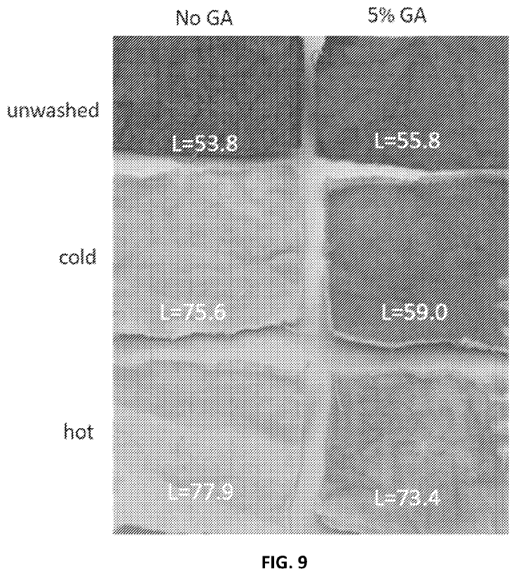

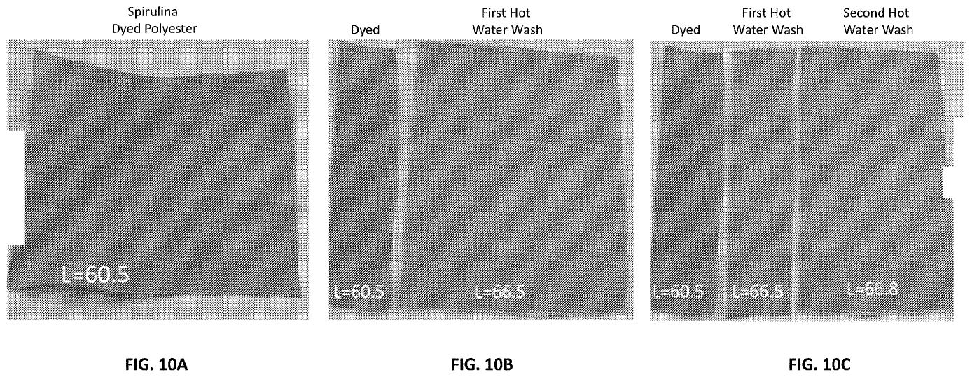

The darkness of the polyester fabrics after dyeing with spirulina is shown in FIG. 10A. The darkness of the spirulina dyed polyester fabric was comparable to dyed cotton fabrics measured by Hunters Scale (Hunter L-number of 60.5 before washing). However, hot wash using commercial detergent product showed some loss of the color (FIG. 10B) in the first hot water wash, i.e., L-66.5 and the subsequent second hot water wash did not show any loss of the darkness (FIG. 10C). Some loss of the darkness in the first wash could be from any unbound color, which was on the surface of the fabric. The results of this example demonstrate that synthetic fabric can be effectively dyed using the low/ambient temperature textile dyeing process provided herein.

### Example 11: Determine Effect of Dyeing Agent Concentration on Fabric Darkness After Hot Water Washing

### Purpose

The purpose of this experiment was to determine the effect of increasing dyeing agent (spirulina) concentrations on the final darkness of a cotton fabric after dyeing using the textile dyeing process of the present disclosure.

### Methods

The effect of different concentrations of spirulina was studied on the darkness of spirulina dyed cotton fabric. In a typical experiment, three pieces of cotton fabric, 2-inch square were taken and soaked together in 9 milliliters of 5% PEI solution for 30 minutes at room temperature. The PEI-soaked cotton fabrics were taken out from PEI soaking and squeezed to remove the excess of PEI solution from the fabrics and moved to a 9-milliliter solution containing either 1.0%, 2.5%, or 5% w/v of a spirulina solution. The pH of the solution was adjusted to pH 8.6 after 15 minutes and then continued soaking for a total of 2 hours at room temperature. The spirulina dyed cotton fabrics were removed from spirulina solutions and squeezed to remove the excess of spirulina solution from the fabric and then soaked in 9 milliliters of 10% tannic acid for 30 minutes. After the tannic acid treatment, the dyed fabrics were transferred to 5% GA for finishing the dyeing process. The dyed cotton fabrics were then squeezed to remove excess of solution and rinsed with cold water before air drying at room temperature. The dried swatches were then subjected to hot water wash, as described in Example 5.

### Results and Conclusion

The results of this experiment, shown in Table 2, demonstrated that increasing the concentration of dyeing agent (spirulina) during soaking resulted in increased darkness of the dyed fabric (decreasing L number). Additionally, a hot water wash of the dyed fabric did not show a significant loss of the darkness.

TABLE 2
Effect of spirulina concentrations on the darkness
of dyed cotton fabric before and after hot wash.

| | | | Hot Water Wash | |
|---|---|---|---|---|
| Number | Spirulina | Before Washing | First wash | Second Wash |
| 1 | 1 | L = 67.4 | L = 66.1 | L = 69 |
| 2 | 2.5 | L = 67.4 | L = 59.8 | L = 59.2 |
| 3 | 5 | L = 56.5 | L = 59.9 | L = 58.3 |

### Example 12: Scale Up Textile Dyeing Process

### Purpose

The purpose of this experiment was to scale the method and determine the effect on the final darkness of a cotton fabric after dyeing using the textile dyeing process of the present disclosure.

### Methods

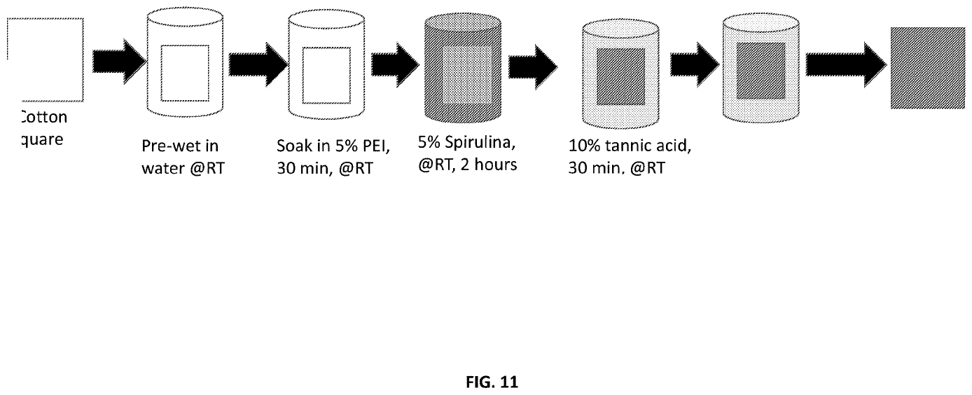

The textile dyeing process described in FIG. 11 was used to dye a generic white cotton fabric. In a typical experiment, a 12-inch square was taken and pre-wet in water at room temperature. The pre-wet soaked cotton fabric was then soaked together in 300 milliliters of 5% PEI solution, (10,000 MW) pH 11, for 30 minutes at room temperature. The PEI-soaked cotton fabric was taken out from PEI soaking and squeezed to remove the excess of PEI solution from the fabric and moved to a solution containing 300 milliliters of 5% w/v of a spirulina solution pH 6 and soaked for a total of 2 hours at room temperature. The spirulina dyed cotton fabric was removed from the spirulina solution and squeezed to remove the excess of spirulina solution from the fabric and then soaked in 300 milliliters of 10% tannic acid, pH 4.5, for 30 minutes. After the tannic acid treatment, the dyed fabric was transferred to 300 milliliters of 5% GA, pH 5, for finishing the dyeing process. The dyed cotton fabric was then squeezed to remove excess of solution and rinsed with cold water before air drying at room temperature. The dried swatches were then subjected to hot water wash, as described in Example 5.

### Results and Conclusion

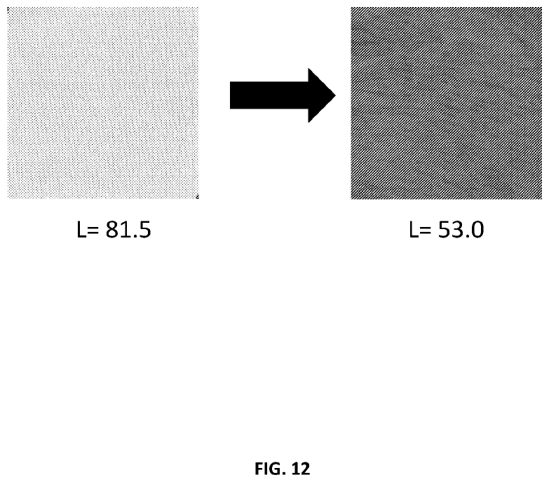

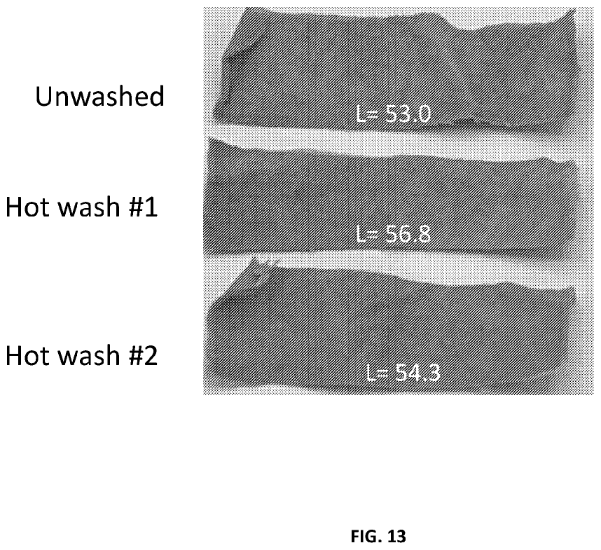

The results of this experiment, shown in FIG. 12 and FIG. 13, demonstrated that scaling the method resulted in consistent darkness of the dyed fabric (decreasing L number) compared to previous experiments. Likewise, the hot water wash of the dyed fabric did not show a significant loss of the darkness.

### Example 13: Hair Dyeing Process

### Purpose

The purpose of this experiment was to alter the dyeing method described herein to be useful in dyeing hair.

### Methods

A blonde hair swatch (Rinboool hair swatch purchased from Amazon) was dyed following the low-temperature dyeing process for textile as disclosed previously herein (e.g., Examples 5 or 10 and FIG. 1A).

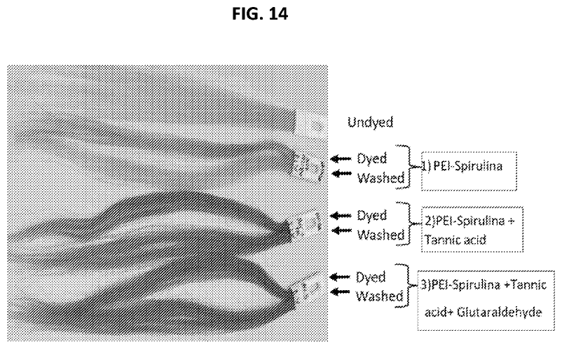

Analysis was completed to determine the critical reagents (all three steps, i.e., (1) PEI-Spirulina, (2) PEI-Spirulina and tannic acid, and (3) finally PEI-Spirulina+tannic acid+Glutaraldehyde) in the hair dyeing process. The soaking time for each bath was reduced to 10 minutes to minimize processing time. In this experiment, three swatches were pre-wet with DI water and squeezed to remove the excess. From there, one Swatch was soaked in the 0.5% PEI-5% Spirulina solution for 10 minutes. After soaking, the excess was removed. The Swatch was rinsed with cool water and allowed to dry overnight. The second Swatch was soaked in 0.5% PEI-5% Spirulina for 10 minutes. The excess was removed from the hair and then submerged in a bath of 10% tannic acid for 10 minutes. That means the second Swatch did not get any glutaraldehyde treatment. Again, after soaking, the Swatch was rinsed with cold water and allowed to dry overnight. The third swatch went through all three reagents including 10-minute soaking in 0.5% PEI-5% Spirulina solution, 10% tannic acid, and 5% glutaraldehyde. Upon completion of the dyeing process, the Swatch was rinsed with cool water and allowed to dry overnight. The next day all Swatches were washed with shampoo and conditioner and allowed to dry. A photo of the resulting hair color can be seen in FIG. 14.

### Conclusions

As can be seen in FIG. 14, hair swatches dyed under condition (1) (i.e., PEI-Spirulina) had very little dye taken up by the hair, but the dyed hair lost all the color after washing with shampoo. In contrast, hair swatches dyed under condition (2) (i.e., PEI-Spirulina+tannic acid) or (3) (i.e., PEI-Spirulina+tannic acid+glutaraldehyde) showed very little difference in the color after dyeing, which was more than under condition (1). However, following washing, the swatch that was not soaked in glutaraldehyde (i.e., condition (3)) lost the blue color.

### NUMBERED EMBODIMENTS

Other subject matter contemplated by the present disclosure is set out in the following numbered embodiments:

### Embodiment 1

A method of dyeing a fiber, comprising:
a) contacting a fiber with a functionalizing agent comprising a poly-ionic polymer;
b) contacting the fiber with a dyeing agent comprising a protein bonded light harvesting chromophore;
c) contacting the fiber with a depositing agent; and
d) contacting the fiber with a cross-linking agent;
   wherein a dyed fiber is obtained.

### Embodiment 2

The method of embodiment 1, wherein the fiber is a natural fiber.

### Embodiment 3

The method of any one of embodiments 1 to 2, wherein the fiber is a cellulosic fiber.

### Embodiment 4

The method of any one of embodiments 1 to 3, wherein the fiber is a cotton, hemp, or jute fiber.

### Embodiment 5

The method of any one of embodiments 1 to 4, wherein the fiber is a protein fiber.

### Embodiment 6

The method of any one of embodiments 1 to 5, wherein the fiber is a silk or wool fiber.

### Embodiment 7

The method of any one of embodiments 1 to 6, wherein the fiber is a synthetic fiber.

### Embodiment 8

The method of any one of embodiments 1 to 7, wherein the fiber is a rayon, nylon, or polyester fiber.

### Embodiment 9

The method of any one of embodiments 1 to 8, wherein the fiber is comprised within a textile.

### Embodiment 10

The method of any one of embodiments 1 to 9, wherein the fiber is comprised within a fabric.

### Embodiment 11

The method of any one of embodiments 1 to 10, wherein the fiber is comprised within a garment.

### Embodiment 12

The method of any one of embodiments 1 to 11, wherein the poly-ionic polymer comprises a poly cationic polymer.

### Embodiment 13

The method of any one of embodiments 1 to 12, wherein the poly-ionic polymer comprises a poly cationic polymer comprising a positively charged functional group.

### Embodiment 14

The method of any one of embodiments 1 to 13, wherein the poly-ionic polymer is selected from the group consisting of: polyethyleneimine, chitosan, derivative of polyacrylamide, carboxy methyl cellulose (CMC), and diethyl aminoethyl cellulose (DEAE-Cellulose).

### Embodiment 15

The method of any one of embodiments 1 to 14, wherein the poly-ionic polymer is polyethyleneimine.

### Embodiment 16

The method of any one of embodiments 1 to 15, wherein the poly-ionic polymer comprises a poly anionic polymer.

### Embodiment 17

The method of any one of embodiments 1 to 16, wherein the poly-ionic polymer comprises a poly anionic polymer comprising a negatively charged functional group.

### Embodiment 18

The method of any one of embodiments 1 to 17, wherein the poly-ionic polymer is selected from the group consisting of: derivative of polyacrylamide, carboxy methyl cellulose (CMC), and diethyl aminoethyl cellulose (DEAE-Cellulose).

### Embodiment 19

The method of any one of embodiments 1 to 18, wherein the poly-ionic polymer is present at a concentration of about 0.01 to 20%.

### Embodiment 20

The method of any one of embodiments 1 to 19, wherein the poly-ionic polymer is present at a concentration of about 0.05 to 5%.

### Embodiment 21

The method of any one of embodiments 1 to 20, wherein the poly-ionic polymer is present at a concentration of about 0.2 to 0.5%.

### Embodiment 22

The method of any one of embodiments 1 to 21, wherein the fiber is contacted with the functionalizing agent comprising a poly-ionic polymer for about 5 minutes or less.

### Embodiment 23

The method of any one of embodiments 1 to 22, wherein the fiber is contacted with the functionalizing agent comprising a poly-ionic polymer for at least about 1 minute.

### Embodiment 24

The method of any one of embodiments 1 to 23, wherein the fiber is contacted with the functionalizing agent comprising a poly-ionic polymer for at least about 10 minutes.

### Embodiment 25

The method of any one of embodiments 1 to 24, wherein the fiber is contacted with the functionalizing agent comprising a poly-ionic polymer for at least about 30 minutes.

### Embodiment 26

The method of any one of embodiments 1 to 25, wherein the fiber is contacted with the functionalizing agent comprising a poly-ionic polymer for at least about 60 minutes.

### Embodiment 27

The method of any one of embodiments 1 to 26, wherein the fiber is contacted with the functionalizing agent comprising a poly-ionic polymer for at least about 120 minutes.

### Embodiment 28

The method of any one of embodiments 1 to 27, wherein the fiber is contacted with the functionalizing agent comprising a poly-ionic polymer for at least about 24 hours.

### Embodiment 29

The method of any one of embodiments 1 to 28, wherein the protein bonded light harvesting chromophore comprises a tetra pyrrole ring.

### Embodiment 30

The method of any one of embodiments 1 to 29, wherein the protein bonded light harvesting chromophore comprises a modified tetra pyrrole ring.

### Embodiment 31

The method of any one of embodiments 1 to 30, wherein the protein bonded light harvesting chromophore comprises a modified tetra pyrrole ring, wherein the tetra pyrrole ring has a different light harvest capacity, as compared to an unmodified tetra pyrrole ring.

### Embodiment 32

The method of any one of embodiments 1 to 31, wherein the protein bonded light harvesting chromophore comprises a phycobiliprotein.

### Embodiment 33

The method of any one of embodiments 1 to 32, wherein the protein bonded light harvesting chromophore is produced by a green or blue algae.

### Embodiment 34

The method of any one of embodiments 1 to 33, wherein the protein bonded light harvesting chromophore is produced by a Spirulina sp.

### Embodiment 35

The method of any one of embodiments 1 to 34, wherein the protein bonded light harvesting chromophore comprises a cyanobacteriochrome.

### Embodiment 36

The method of any one of embodiments 1 to 35, wherein the protein bonded light harvesting chromophore is produced by a Cyanobacteria.

### Embodiment 37

The method of any one of embodiments 1 to 36, wherein the depositing agent comprises an acid solution.

### Embodiment 38

The method of any one of embodiments 1 to 37, wherein the depositing agent comprises an acid solution comprising tannic acid.

### Embodiment 39

The method of any one of embodiments 1 to 38, wherein the depositing agent comprises an acid solution comprising citric acid.

### Embodiment 40

The method of any one of embodiments 1 to 39, wherein the depositing agent comprises a mordant.

### Embodiment 41

The method of any one of embodiments 1 to 40, wherein the depositing agent comprises a metal salt.

### Embodiment 42

The method of any one of embodiments 1 to 41, wherein the depositing agent is present at a concentration of about 0.01 to 20%.

### Embodiment 43

The method of any one of embodiments 1 to 42, wherein the depositing agent is present at a concentration of about 5% to 15%.

### Embodiment 44

The method of any one of embodiments 1 to 43, wherein the cross-linking agent comprises a bi-functional protein.

### Embodiment 45

The method of any one of embodiments 1 to 44, wherein the cross-linking agent comprises glutaraldehyde.

### Embodiment 46

The method of any one of embodiments 1 to 45, wherein the cross-linking agent is present at a concentration of about 0.1 to 10%.

### Embodiment 47

The method of any one of embodiments 1 to 46, wherein the cross-linking agent is present at a concentration of about 0.1 to 5%.

### Embodiment 48

The method of any one of embodiments 1 to 47, wherein the cross-linking agent is present at a concentration of about 0.1 to 0.5%.

### Embodiment 49

The method of any one of embodiments 1 to 48, further comprising: e) contacting the fiber with a fixing agent.

### Embodiment 50

The method of any one of embodiments 1 to 49, wherein the fixing agent comprises a mordant.

### Embodiment 51

The method of any one of embodiments 1 to 50, wherein the fixing agent comprises a metal salt.

### Embodiment 52

The method of any one of embodiments 1 to 51, wherein the fixing agent comprises nickel sulphate.

### Embodiment 53

The method of any one of embodiments 1 to 52, wherein the fixing agent comprises nickel sulphate present at a concentration of about 0.1 to 5%.

### Embodiment 54

The method of any one of embodiments 1 to 53, wherein the fixing agent comprises nickel sulphate present at a concentration of about 0.1 to 1%.

### Embodiment 55

The method of any one of embodiments 1 to 54, further comprising: rinsing unbound protein bonded light harvesting chromophore from the fiber.

### Embodiment 56

The method of any one of embodiments 1 to 55, further comprising: repeating any of step a)-e) one or more times.

### Embodiment 57

The method of any one of embodiments 1 to 56, wherein steps a)-e) are conducted at ambient temperature.

### Embodiment 58

The method of any one of embodiments 1 to 57, wherein steps a)-e) are conducted at a temperature of 40° C. or below.

### Embodiment 59

The method of any one of embodiments 1 to 58, wherein steps a)-e) are conducted at a temperature of 30° C. or below.

### Embodiment 60

The method of any one of embodiments 1 to 59, wherein steps a)-e) are conducted at a temperature of between about 15° C. to 30° C.

### Embodiment 61

The method of embodiments 1 to 60, wherein the dyeing agent is present at a concentration of about 0.1 to 15%.

### Embodiment 62

The method of embodiments 1 to 61, wherein the dyeing agent is present at a concentration of about 1 to 10%.

### Embodiment 63

The method of embodiments 1 to 62, wherein the dyeing agent is present at a concentration of about 5%.

### Embodiment 64

A fiber dyed by the method of any one of embodiments 1 to 63.

### Embodiment 65

A thread comprising the dyed fiber of any one of embodiments 1 to 64.

### Embodiment 66

A woven thread comprising the dyed fiber of any one of embodiments 1 to 64.

### Embodiment 67

A non-woven thread comprising the dyed fiber of any one of embodiments 1 to 64.

### Embodiment 68

A yarn comprising the dyed fiber of any one of embodiments 1 to 64.

### Embodiment 69

A textile comprising the dyed fiber of any one of embodiments 1 to 64.

### Embodiment 70

A fabric comprising the dyed fiber of any one of embodiments 1 to 64.

### Embodiment 71

A garment comprising the dyed fiber of any one of embodiments 1 to 64.

### Embodiment 72

The method of any one of embodiments 1 to 71, wherein the obtained dyed fiber has a stable color.

### Embodiment 73

The method of any one of embodiments 1 to 72, wherein the obtained dyed fiber has a score of 62 or below on the Hunter-L scale.

### Embodiment 74

The method of any one of embodiments 1 to 73, wherein the obtained dyed fiber has a stable score, as measured on the Hunter-L scale.

### Embodiment 75

The method of any one of embodiments 1 to 74, wherein the obtained dyed fiber has a stable score, as measured on the Hunter-L scale, such that the dyed fiber's Hunter-L score does not fluctuate more than 10 units after washing in water.

### Embodiment 76

The method of any one of embodiments 1 to 75, wherein the obtained dyed fiber has a stable score, as measured on the Hunter-L scale, such that the dyed fiber's Hunter-L score does not fluctuate more than 5 units after washing in water.

### Embodiment 77

The method of any one of embodiments 1 to 76, wherein the obtained dyed fiber has a stable color, as compared to a dyed fiber that was not subject to a step a) of contacting with a functionalizing agent comprising a poly-ionic polymer.

### Embodiment 77.1

The method of any one of embodiments 1 to 77, further comprising pre-wetting the fiber before step (a)

### Embodiment 77.2

The method of embodiment 77.1, wherein the pre-wetting comprises contacting the fiber with water.

### Embodiment 77.3

The method of any one of embodiments 1 to 77.2, wherein the fiber is hair.

### Embodiment 78

A composition comprising a fiber, wherein the fiber has a protein bonded light harvesting chromophore and a cross-linking agent attached thereto.

### Embodiment 79

The composition of embodiment 78, wherein the protein bonded light harvesting chromophore comprises a tetra pyrrole ring.

### Embodiment 80

The composition of embodiments 78 or 79, wherein the protein bonded light harvesting chromophore comprises a modified tetra pyrrole ring.

### Embodiment 81

The composition of any one of embodiments 78 to 80, wherein the protein bonded light harvesting chromophore comprises a modified tetra pyrrole ring, wherein the tetra pyrrole ring has a different light harvest capacity, as compared to an unmodified tetra pyrrole ring.

### Embodiment 82

The composition of any one of embodiments 78 to 81, wherein the protein bonded light harvesting chromophore comprises a phycobiliprotein.

### Embodiment 83

The composition of any one of embodiments 78 to 82, wherein the protein bonded light harvesting chromophore is produced by a green or blue algae.

### Embodiment 84

The composition of any one of embodiments 78 to 83, wherein the protein bonded light harvesting chromophore is produced by a Spirulina sp.

### Embodiment 85

The composition of any one of embodiments 78 to 84, wherein the protein bonded light harvesting chromophore comprises a cyanobacteriochrome.

### Embodiment 86

The composition of any one of embodiments 78 to 85, wherein the protein bonded light harvesting chromophore is produced by a Cyanobacteria.

### Embodiment 87

The composition of any one of embodiments 78 to 86, wherein the cross-linking agent comprises a bi-functional protein.

### Embodiment 88

The composition of any one of embodiments 78 to 87, wherein the cross-linking agent comprises glutaraldehyde.

### Embodiment 89

The composition of any one of embodiments 78 to 88, wherein the cross-linking agent is attached to primary, secondary or tertiary amino groups.

### Embodiment 90

The method of any one of embodiments 1 to 64, wherein the fiber is hair.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgement or any form of suggestion that they constitute valid prior art or form part of the common general knowledge in any country in the world.

1. A method of dyeing a fiber, comprising: a) contacting a fiber with a functionalizing agent comprising a poly-ionic polymer; b) contacting the fiber with a dyeing agent comprising a protein bonded light harvesting chromophore; c) contacting the fiber with a depositing agent; and d) contacting the fiber with a cross-linking agent, wherein a dyed fiber is obtained.

2. The method of claim 1, wherein the fiber is a natural fiber.

3. The method of claim 1, wherein the fiber is a cellulosic fiber.

4. The method of claim 1, wherein the fiber is a cotton, hemp, or jute fiber.

5. The method of claim 1, wherein the fiber is a protein fiber.

6. The method of claim 1, wherein the fiber is a silk or wool fiber.

7. The method of claim 1, wherein the fiber is a synthetic fiber.

8. The method of claim 1, wherein the fiber is a rayon, nylon, or polyester fiber.

9. The method of claim 1, wherein the fiber is comprised within a textile.

10. The method of claim 1, wherein the fiber is comprised within a fabric.

11. The method of claim 1, wherein the fiber is comprised within a garment.

12. The method of claim 1, wherein the poly-ionic polymer comprises a poly cationic polymer.

13. The method of claim 1, wherein the poly-ionic polymer comprises a poly cationic polymer comprising a positively charged functional group.

14. The method of claim 1, wherein the poly-ionic polymer is selected from the group consisting of: polyethyleneimine, chitosan, derivative of polyacrylamide, carboxy methyl cellulose (CMC), and diethyl aminoethyl cellulose (DEAE-Cellulose).

15. The method of claim 1, wherein the poly-ionic polymer is polyethyleneimine.

16. The method of claim 1, wherein the poly-ionic polymer comprises a poly anionic polymer.

17. The method of claim 1, wherein the poly-ionic polymer comprises a poly anionic polymer comprising a negatively charged functional group.

18. The method of claim 1, wherein the poly-ionic polymer is selected from the group consisting of: derivative of polyacrylamide, carboxy methyl cellulose (CMC), and diethyl aminoethyl cellulose (DEAE-Cellulose).

19. The method of claim 1, wherein the poly-ionic polymer is present at a concentration of about 0.01 to 20%.

20. The method of claim 1, wherein the poly-ionic polymer is present at a concentration of about 0.05 to 5%.

21.-93. (canceled)
